(19) United States
(12) Patent Application Publication
(10) Pub. No.: US 2024/0226112 A1
(43) Pub. Date: Jul. 11, 2024

(54) CERTAIN N-(1-CYANO-2-PHENYLETHYL)-1,4-OXAZEPANE-2-CARBOXAMIDES FOR TREATING CYSTIC FIBROSIS

(71) Applicant: Insmed Incorporated, Bridgewater, NJ (US)

(72) Inventors: Helen Hui USANSKY, Bridgewater, NJ (US); Ariel TEPER, Bridgewater, NJ (US); Marcela Martha VERGARA, Bridgewater, NJ (US); Andrea Lynn MAES, Bridgewater, NJ (US)

(21) Appl. No.: 18/288,338

(22) PCT Filed: Apr. 29, 2022
(86) PCT No.: PCT/US22/27026

§ 371 Date: Oct. 25, 2023

Related U.S. Application Data
(60) Provisional application No. 63/181,817, filed on Apr. 29, 2021.

(51) Int. Cl. A61K 31/553; A61K 9/20; A61K 45/06

(52) U.S. Cl. CPC A61K 31/553; A61K 9/2009; A61K 9/2013; A61K 9/2054; A61K 9/2059; A61K 45/06

(57) ABSTRACT

The present disclosure relates to methods for treating cystic fibrosis with compositions comprising an effective amount of certain N-(1-cyano-2-phenylethyl)-1,4-oxazepane-2-carboxamide compounds of Formula (I), including pharmaceutically acceptable salts thereof,

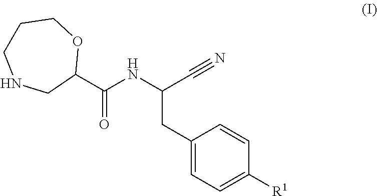

that reversibly inhibit dipeptidyl peptidase 1 (DPP1) activity. Methods provided herein are useful for increasing the lung function in a patient, and/or improving the patient's quality of life (QOL) assessed by the cystic fibrosis questionnaire-revised (CFQ-R). In one embodiment, the compound of Formula (I) is brensocatib.

### CROSS REFERENCE TO RELATED APPLICATION

This application is a National Stage of International Patent Application Number PCT/US2022/027026, filed Apr. 29, 2022, which claims priority from U.S. Provisional Application No. 63/181,817, filed Apr. 29, 2021, the disclosure of each of which is incorporated by reference herein in its entirety.

### BACKGROUND OF THE INVENTION

Cystic fibrosis (CF), an autosomal recessive disorder, is a disease of exocrine gland function that involves multiple organ systems, but primarily results in chronic respiratory infections, pancreatic enzyme insufficiency, and associated complications. Defects in the CF transmembrane conductance regulator (CFTR) gene cause abnormalities of cyclic adenosine monophosphate-regulated chloride transport across epithelial cells on mucosal surfaces.

Defective CFTR results in decreased secretion of chloride and increased reabsorption of sodium and water across epithelial cells. The resultant decreased hydration of mucus results in viscous mucus, which promotes infection and inflammation. Secretions in the respiratory tract, pancreas, gastrointestinal tract, sweat glands, and other exocrine tissues have increased viscosity, making them difficult to clear.

Worldwide incidence of CF varies from 1 per 377 live births (England) to 1 per 90,000 Asian live births in Hawaii. It is the most common lethal hereditary disease among Caucasians in the U.S. (1 case per 2,500-3,500 births), with lower incidence among African Americans (1 in 17,000 births) and Asian Americans (1 in 31,000 births).

In 2019, there were 31,199 persons diagnosed with CF in the United States, and of these, the majority were Caucasian (93.4%) and male (51.8%). The annual mortality rate (per 100) was 1.2%, with a median age at death of 32.4 years. However, because of improvements in the treatment for CF, the median predicted survival age has continued to increase since 1988: from approximately 29 years to 48.4 years (95% CI: 45.9-51.5 years) in 2019.

Pulmonary outcomes are a key measure of CF health. Pulmonary exacerbations (PEs) that require intravenous antibiotic treatment in the hospital or at home, are associated with morbidity, mortality, and decreased quality of life. Uncontrolled PEs often result in prolong hospitalization and consequently permanent damage to the lung which manifests as lung function decline. The most severe manifestation of CF (and the most frequent cause of death or lung transplant) is chronic lung disease with the presence of bilateral disseminated bronchiectasis, characterized by chronic lung infection, particularly with Staphylococcus aureus and Pseudomonas aeruginosa, and excessive inflammation, declining lung function, and eventually respiratory insufficiency.

Currently available pharmacologic treatment for CF PEs includes inhaled antibiotics (tobramycin, aztreonam, and colistin), inhaled corticosteroids, leukotriene modifiers, and inhaled beta agonists. Despite improvement in pulmonary function over the years, there has not been a marked change in the proportion of individuals with CF who are treated with antibiotics for PEs. Approximately 45% of patients 18 years and older were treated with IV antibiotics for pulmonary exacerbation from 2005 to 2019.

The present invention addresses the need for a therapy effective for the treatment of cystic fibrosis.

### SUMMARY OF THE INVENTION

In one aspect, a method for treating a CF patient is provided. The method comprises, in one embodiment, administering to a patient in need of treatment, for an administration period, a pharmaceutical composition comprising an effective amount of a compound of Formula (I), or a pharmaceutically acceptable salt of a compound of Formula (I):

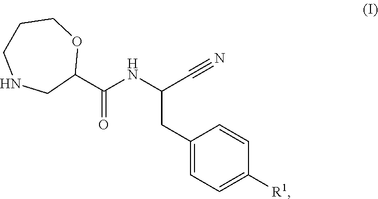

wherein,
R¹ is

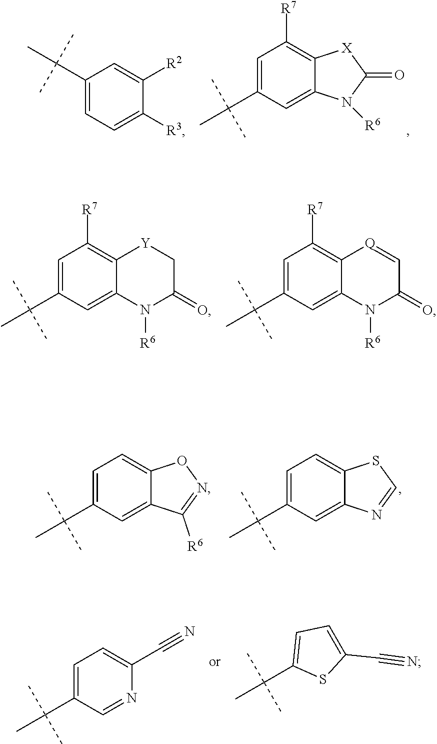

R² is hydrogen, F, Cl, Br, OSO₂C₁₋₃alkyl, or C₁₋₃alkyl;
R³ is hydrogen, F, Cl, Br, CN, CF₃, SO₂C₁₋₃alkyl, CONH₂ or SO₂NR⁴R⁵,
wherein R⁴ and R⁵ together with the nitrogen atom to which they are attached form an azetidine, pyrrolidine or piperidine ring;
X is O, S or CF₂;
Y is O or S;
Q is CH or N;
R⁶ is C₁₋₃alkyl, wherein the C₁₋₃alkyl is optionally substituted by 1, 2 or 3 F and optionally by one substituent selected from OH, OC₁₋₃alkyl, N(C₁₋₃alkyl)₂, cyclopropyl, or tetrahydropyran; and
R⁷ is hydrogen, F, Cl or CH₃.

In the methods provided herein, the treating comprises (i) improving the lung function of the patient, as compared to the lung function of the patient prior to the administration period; (ii) improving the patient's quality of life (QOL) assessed by the cystic fibrosis questionnaire-revised (CFQ-R), as compared to the patient's QOL prior to the administration period; or (iii) both (i) and (ii).

In one embodiment, the compound of Formula (I) is an S,S diastereomer. In other words, the compound of Formula (I) has the following stereochemistry:

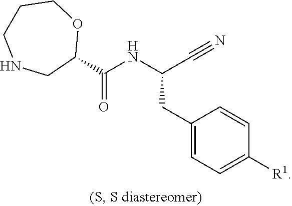

The other diastereomeric forms are also contemplated by the present invention. For example, in one embodiment, the compound of Formula (I) is the R,R diastereomer:

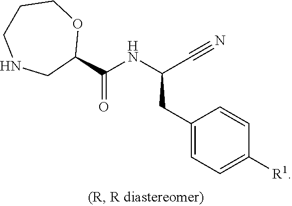

In another embodiment, the compound of Formula (I) is the R,S diastereomer:

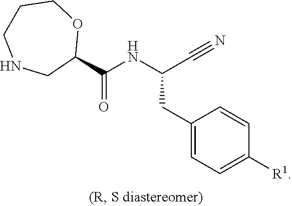

In even another embodiment, the compound of Formula (I) is the S,R diastereomer:

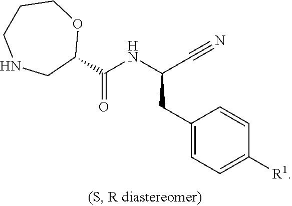

In one embodiment of the method for treating CF in a patient in need of treatment, the pharmaceutical composition comprises an effective amount of (2S)—N-{(1S)-1-cyano-2-[4-(3-methyl-2-oxo-2,3-dihydro-1,3-benzoxazol-5-yl)phenyl]ethyl}-1,4-oxazepane-2-carboxamide, referred to herein by its international nonproprietary name (INN), brensocatib (and formerly known as INS1007 and AZD7986),

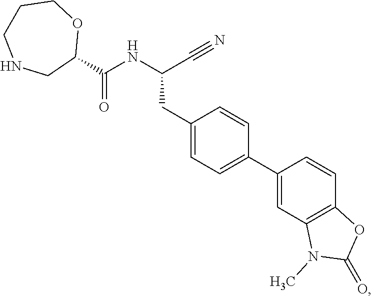 [SMILES: Cn1c(=O)oc2ccc(-c3ccc(C[C@@H](C#N)NC(=O)[C@@H]4CNCCCO4)cc3)cc21]

or a pharmaceutically acceptable salt thereof.

In one embodiment of the method for treating CF in a patient in need of treatment, the pharmaceutical composition comprises an effective amount of (2S)—N-{(1R)-1-Cyano-2-[4-(3-methyl-2-oxo-2,3-dihydro-1,3-benzoxazol-5-yl)phenyl]ethyl}-1,4-oxazepane-2-carboxamide:

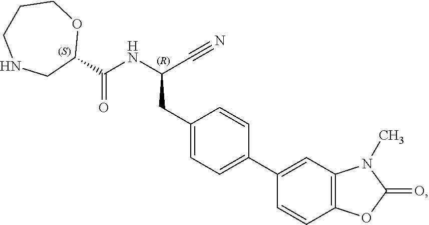

or a pharmaceutically acceptable salt thereof.

In one embodiment of the method for treating CF in a patient in need of treatment, the pharmaceutical composition comprises an effective amount of (2R)—N-{(1S)-1-Cyano-2-[4-(3-methyl-2-oxo-2,3-dihydro-1,3-benzoxazol-5-yl)phenyl]ethyl}-1,4-oxazepane-2-carboxamide:

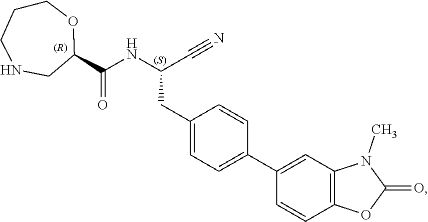

or a pharmaceutically acceptable salt thereof.

In one embodiment of the method for treating CF in a patient in need of treatment, the pharmaceutical composition comprises an effective amount of (2R)—N-{(1R)-1-Cyano-2-[4-(3-methyl-2-oxo-2,3-dihydro-1,3-benzoxazol-5-yl)phenyl]ethyl}-1,4-oxazepane-2-carboxamide:

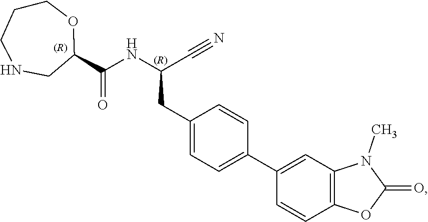

or a pharmaceutically acceptable salt thereof.

In one embodiment of the method, the patient is administered the composition once daily. In another embodiment, the patient is administered the composition twice daily, or every other day, or once a week. Administration, in one embodiment, is via the oral route. In a further embodiment, the compound of Formula (I) is present at about 10 mg, about 25 mg, about 40 mg, or about 65 mg in the composition and the administration is carried out once daily. In a further embodiment, the compound of Formula (I) is brensocatib.

In one embodiment of the methods provided herein, treating comprises improving the lung function of the patient. The improvement in the patient's lung function, in one embodiment, comprises increasing the patient's forced expiratory volume in one second (FEV₁), as compared to the patient's FEV₁ prior to the administration period. In one embodiment, the increase in FEV₁ is an increase in pre-bronchodilator FEV₁. In another embodiment, the increase in FEV₁ is an increase in post-bronchodilator FEV₁. In one embodiment, the patient's FEV₁ is increased by about 5%, by about 10%, by about 15%, by about 20%, by about 25%, by about 30%, by about 35%, by about 40%, by about 45%, or by about 50%. In another embodiment, the patient's FEV₁ is increased by at least about 5%, by at least about 10%, by at least about 15%, by at least about 20%, by at least about 25%, by at least about 30%, by at least about 35%, by at least about 40%, by at least about 45%, or by at least about 50%. In yet another embodiment, the patient's FEV₁ is increased by about 5% to about 50%, by about 5% to about 40%, by about 5% to about 30%, by about 5% to about 20%, by about 10% to about 50%, by about 15% to about 50%, by about 20% to about 50%, or by about 25% to about 50%. In even another embodiment, the patient's FEV₁ is increased about 25 mL to about 500 mL, or about 25 mL to about 250 mL.

In another embodiment, the improvement in the lung function of the patient comprises increasing the patient's percent predicted forced expiratory volume in one second (ppFEV₁) compared to the patient's ppFEV₁ prior to the administration period. In one embodiment, the increase in ppFEV₁ is an increase in pre-bronchodilator ppFEV₁. In another embodiment, the increase in ppFEV₁ is an increase in post-bronchodilator ppFEV₁. In one embodiment, the patient's ppFEV₁ is increased by about 1%, by about 2%, by about 3%, by about 4%, by about 5%, by about 6%, by about 7%, by about 8%, by about 9%, by about 10%, by about 11%, by about 12%, by about 13%, by about 14%, by about 15%, by about 16%, by about 17%, by about 18%, by about 19%, by about 20%, by about 25%, by about 30%, by about 35%, by about 40%, by about 45%, by about 50%, by about 55%, by about 60%, by about 65%, by about 70%, by about 75%, by about 80%, by about 85%, or by about 90%. In another embodiment, the patient's ppFEV₁ is increased by at least about 5%, by at least about 10%, by at least about 15%, by at least about 20%, by at least about 25%, by at least about 30%, by at least about 35%, by at least about 40%, by at least about 45%, or by at least about 50%. In another embodiment, the patient's ppFEV₁ is increased by about 5% to about 50%, by about 5% to about 40%, by about 5% to about 30%, by about 5% to about 20%, by about 10% to about 50%, by about 15% to about 50%, by about 20% to about 50%, or by about 25% to about 50%. In one embodiment, the patient's ppFEV₁ is about 40% or more (e.g., from about 40% to about 90%) prior to the administration period.

In another embodiment, the improvement in the lung function of the patient comprises increasing the patient's forced vital capacity (FVC), as compared to the patient's FVC prior to the administration period. In one embodiment, the increase in FVC is an increase in pre-bronchodilator FVC. In another embodiment, the increase in FVC is an increase in post-bronchodilator FVC. In one embodiment, the patient's FVC is increased by about 1%, by about 2%, by about 3%, by about 4%, by about 5%, by about 6%, by about 7%, by about 8%, by about 9%, by about 10%, by about 11%, by about 12%, by about 13%, by about 14%, by about 15%, by about 16%, by about 17%, by about 18%, by about 19%, by about 20%, by about 25%, by about 30%, by about 35%, by about 40%, by about 45%, by about 50%, by about 55%, by about 60%, by about 65%, by about 70%, by about 75%, by about 80%, by about 85% or by about 90%. In another embodiment, the patient's FVC is increased by at least about 5%, by at least about 10%, by at least about 15%, by at least about 20%, by at least about 25%, by at least about 30%, by at least about 35%, by at least about 40%, by at least about 45% or by at least about 50%. In even another embodiment, the patient's FVC is increased by about 5% to about 50%, by about 5% to about 40%, by about 5% to about 30%, by about 5% to about 20%, by about 10% to about 50%, by about 15% to about 50%, by about 20% to about 50%, or by about 25% to about 50%.

In another embodiment, improving the lung function of the patient comprises increasing the patient's forced expiratory flow between 25% and 75% of FVC (FEF_{(25-75%)}), as compared to the patient's FEF_{(25-75%)} prior to the administration period. In one embodiment, the increase in FEF_{(25-75%)} is an increase in pre-bronchodilator FEF_{(25-75%)}. In another embodiment, the increase in FEF_{(25-75%)} is an increase in post-bronchodilator FEF_{(25-75%)}. In one embodiment, the patient's FEF_{(25-75%)} is increased by about 1%, by about 2%, by about 3%, by about 4%, by about 5%, by about 6%, by about 7%, by about 8%, by about 9%, by about 10%, by about 11%, by about 12%, by about 13%, by about 14%, by about 15%, by about 16%, by about 17%, by about 18%, by about 19%, by about 20%, by about 25%, by about 30%, by about 35%, by about 40%, by about 45%, by about 50%, by about 55%, by about 60%, by about 65%, by about 70%, by about 75%, by about 80%, by about 85% or by about 90%. In another embodiment, the patient's FEF_{(25-75%)} is increased by at least about 5%, by at least about 10%, by at least about 15%, by at least about 20%, by at least about 25%, by at least about 30%, by at least about 35%, by at least about 40%, by at least about 45%, or by at least about 50%. In even another embodiment, the patient's FEF_{(25-75%)} is increased by about 5% to about 50%, by about 5% to about 40%, by about 5% to about 30%, by about 5% to about 20%, by about 10% to about 50%, by about 15% to about 50%, by about 20% to about 50%, or by about 25% to about 50%.

In another embodiment, improving the lung function of the patient comprises increasing the patient's peak expiratory flow rate (PEFR), as compared to the patient's PEFR prior to the administration period. In one embodiment, the increase in PEFR is an increase in pre-bronchodilator PEFR. In another embodiment, the increase in PEFR is an increase in post-bronchodilator PEFR. In one embodiment, the patient's PEFR is increased by about 1%, by about 2%, by about 3%, by about 4%, by about 5%, by about 6%, by about 7%, by about 8%, by about 9%, by about 10%, by about 11%, by about 12%, by about 13%, by about 14%, by about 15%, by about 16%, by about 17%, by about 18%, by about 19%, by about 20%, by about 25%, by about 30%, by about 35%, by about 40%, by about 45%, by about 50%, by about 55%, by about 60%, by about 65%, by about 70%, by about 75%, by about 80%, by about 85% or by about 90%. In another embodiment, the patient's PEFR is increased by at least about 5%, by at least about 10%, by at least about 15%, by at least about 20%, by at least about 25%, by at least about 30%, by at least about 35%, by at least about 40%, by at least about 45% or by at least about 50%. In even another embodiment, the patient's PEFR is increased by about 5% to about 50%, by about 5% to about 40%, by about 5% to about 30%, by about 5% to about 20%, by about 10% to about 50%, by about 15% to about 50%, by about 20% to about 50%, or by about 25% to about 50%.

In another embodiment of a method for treating CF, a patient in need of treatment is administered a composition comprising an effective amount of a compound of Formula (I), or a pharmaceutically acceptable salt thereof, for an administration period. In this embodiment, the treating comprises improving the patient's quality of life (QOL) assessed by the cystic fibrosis questionnaire-revised (CFQ-R), as compared to the patient's QOL assessed by the CFQ-R prior to the administration period. In a further embodiment, the QOL is assessed by a respiratory domain score of the CFQ-R.

In yet another embodiment of a method for treating CF, a patient in need of treatment is administered a composition comprising an effective amount of a compound of Formula (I), or a pharmaceutically acceptable salt thereof for an administration period, and the treating comprises decreasing the sputum and/or blood concentration of an active neutrophil serine protease (NSP) in the patient, as compared to the patient's active NSP sputum and/or blood concentration prior to the administration period. In a further embodiment, the patient's active NSP sputum and/or blood concentration is decreased by about 1%, about 5%, about 10%, about 20%, about 25%, about 30%, about 40%, about 50%, about 60%, about 70%, or about 80%, at least about 1%, at least about 5%, at least about 10%, at least about 20%, at least about 25%, at least about 30%, at least about 40%, at least about 50%, at least about 60%, at least about 70%, or at least about 80%. In one embodiment, the active NSP is active neutrophil elastase (NE). In another embodiment, the active NSP is active proteinase 3 (PR3). In another embodiment, the active NSP is active cathepsin G (CatG).

In yet another embodiment of a method for treating CF, a patient in need of treatment is administered a composition comprising an effective amount of a compound of Formula (I), or a pharmaceutically acceptable salt thereof for an administration period, and the treating comprises decreasing a bacterial infection in the lung of the patient, as compared to the bacterial infection in the lung of the patient prior to the administration period. In one embodiment, the bacterial infection comprises a Pseudomonas infection, e.g., Pseudomonas aeruginosa infection. In another embodiment, the bacterial infection comprises Staphylococcus aureus infection. In a further embodiment, the Staphylococcus aureus infection is a methicillin-resistant Staphylococcus aureus (MRSA) infection. In one embodiment, decreasing the bacterial infection in the lung of the patient comprises decreasing a number of colony forming units (CFUs) of the bacteria present in the patient's sputum, as compared to a number of CFUs of the bacteria present in the patient's sputum prior to the administration period. In one embodiment, the number of CFUs of the bacteria present in the treated patient's sputum is decreased about 1%, about 5%, about 10%, about 20%, about 25%, about 30%, about 40%, about 50%, about 60%, about 70%, about 80%, or about 90%. In another embodiment, the number of CFUs of the bacteria present in the treated patient's sputum is decreased at least about 1%, at least about 5%, at least about 10%, at least about 20%, at least about 30%, at least about 40%, at least about 50%, at least about 60%, at least about 70%, at least about 80%, or at least about 90%. In another embodiment, the number of CFUs of the bacteria present in the treated patient's sputum is decreased by about 5% to about 50%, by about 5% to about 40%, by about 5% to about 30%, by about 5% to about 20%, by about 10% to about 50%, by about 15% to about 50%, by about 20% to about 50%, or by about 25% to about 50%.

In one embodiment of a method for treating CF, a patient in need of treatment is co-administered an antibiotic together with a compound of Formula (I) or its pharmaceutically acceptable salt. In some embodiments, the antibiotic is selected from the group consisting of an aminoglycoside, aztreonam, a carbapenem, a cephalosporin, clofazimine, colistimethate, ethambutol, a lincosamide, a macrolide, an oxazolidinone, a penicillin, a quinolone, a rifamycin, a sulfa, a tetracycline, vancomycin, and a combination thereof. In some embodiments, the antibiotic is selected from the group consisting of amikacin, aztreonam, colistimethate, gentamicin, tobramycin, or a combination thereof. In a further embodiment, the antibiotic is administered to the patient via inhalation.

In one embodiment of a method for treating CF, a patient in need of treatment is co-administered a cystic fibrosis transmembrane conductance regulator (CFTR) modulator together with a compound of Formula (I) or its pharmaceutically acceptable salt. In one embodiment, the patient has not previously been treated with a CFTR modulator. In a further embodiment, the CFTR modulator is one selected from the group consisting of ivacaftor, lumacaftor, tezacaftor, elexacaftor, and a combination thereof.

In one embodiment of the methods provided herein, a patient in need of treatment has previously been treated with a cystic fibrosis transmembrane conductance regulator (CFTR) modulator. In a further embodiment, the method of treating CF comprises administering the CFTR modulator to the patient, together with an effective amount of a compound of Formula (I). In a further embodiment, the CFTR modulator is one selected from the group consisting of ivacaftor, lumacaftor, tezacaftor, elexacaftor, and a combination thereof.

In another embodiment of a method for treating CF, a patient in need of treatment has not previously been treated with a CFTR modulator. In a further embodiment, a patient previously untreated with a CFTR modulator is administered a composition comprising an effective amount of a compound of Formula (I) as monotherapy, i.e., the method excludes administering a CFTR modulator to the patient.

In another aspect, the present disclosure provides the diastereomers of brensocatib disclosed herein, i.e., (2R)—N-{(1R)-1-Cyano-2-[4-(3-methyl-2-oxo-2,3-dihydro-1,3-benzoxazol-5-yl)phenyl]ethyl}-1,4-oxazepane-2-carboxamide (i.e., the R,R isomer), (2S)—N-{(1R)-1-Cyano-2-[4-(3-methyl-2-oxo-2,3-dihydro-1,3-benzoxazol-5-yl)phenyl]ethyl}-1,4-oxazepane-2-carboxamide (i.e., the S,R isomer), and (2R)—N-{(1S)-1-Cyano-2-[4-(3-methyl-2-oxo-2,3-dihydro-1,3-benzoxazol-5-yl)phenyl]ethyl}-1,4-oxazepane-2-carboxamide (i.e., the R,S isomer), and their respective pharmaceutically acceptable salts, as well as mixtures comprising brensocatib, or a pharmaceutically acceptable salt thereof, and one or more of the diastereomers of brensocatib or pharmaceutically acceptable salts thereof. In one embodiment, the mixture comprises brensocatib, or a pharmaceutically acceptable salt thereof, and the R,R isomer, or a pharmaceutically acceptable salt thereof. In another embodiment, the mixture comprises brensocatib, or a pharmaceutically acceptable salt thereof, and the S,R isomer, or a pharmaceutically acceptable salt thereof. In still another embodiment, the mixture comprises brensocatib, or a pharmaceutically acceptable salt thereof, and the R,S isomer, or a pharmaceutically acceptable salt thereof.

### BRIEF DESCRIPTION OF THE FIGURE

FIG. 1 is an illustration of two chiral centers on the backbone of compounds of Formula (I).

FIG. 2 is a schematic diagram of the study design according to Example 1.

### DETAILED DESCRIPTION OF THE INVENTION

Neutrophils contain four main types of granules: (i) azurophilic or primary granules, (ii) specific or secondary granules, (iii) gelatinase or tertiary granules, and (iv) secretory granules. Azurophilic granules are believed to be the first to form during neutrophil maturation in the bone marrow and are characterized by the expression of related neutrophil serine proteases (NSPs): neutrophil elastase (NE), proteinase 3 (PR3), and cathepsin G (CatG). The lysosomal cysteine dipeptidyl peptidase 1 (DPP1) is the proteinase that activates these 3 NSPs by removal of the N-terminal dipeptide sequences from their precursors during azurophilic granule assembly (Pham et al. (2004). J Immunol. 173(12), pp. 7277-7281). DPP1 is broadly expressed in tissues, but is highly expressed in cells of hematopoietic lineage such as neutrophils.

The three NSPs, abundantly secreted into the extracellular environment upon neutrophil activation at inflammatory sites, are thought to act in combination with reactive oxygen species to assist in degradation of engulfed microorganisms inside phagolysosomes. A fraction of the released proteases remains bound in an active form on the external surface of the plasma membrane, so that both soluble and membrane-bound NSPs can regulate the activities of a variety of biomolecules, such as chemokines, cytokines, growth factors, and cell surface receptors. Regulation is thought to occur by either converting the respective biomolecule to an active form or by degrading the biomolecule by proteolytic cleavage. Secreted proteases can stimulate mucus secretion and inhibit mucociliary clearance, but also activate lymphocytes and cleave apoptotic and adhesion molecules (Bank and Ansorge (2001). J Leukoc Biol. 69, pp. 197-206; Pham (2006). Nat Rev Immunol. 6, pp. 541-550; Meyer-Hoffert (2009). Front Biosci. 14, pp. 3409-3418; Voynow et al. (2004). Am J Physiol Lung Cell Mol Physiol. 287, pp. L1293-302; the disclosure of each of which is incorporated by reference in its entirety for all purposes).

The physiological balance between proteases and anti-proteases is required for the maintenance of the lung's connective tissue. For example, an imbalance in favor of proteases can result in lung injury (Umeki et al. (1988). Am J Med Sci. 296, pp. 103-106; Tetley (1993). Thorax 48, pp. 560-565; the disclosure of each of which is incorporated by reference in its entirety for all purposes).

Cystic fibrosis (CF) is caused by abnormalities in the CF transmembrane conductance regulator protein, causing chronic lung infections (particularly with Pseudomonas aeruginosa) and excessive inflammation, and leading to bronchiectasis, declining lung function, respiratory insufficiency and quality of life. The inflammatory process is dominated by neutrophils that produce NE, as well as other destructive NSPs including CatG and PR3, that directly act upon extracellular matrix proteins and play a role in the host response to inflammation, P. aeruginosa infection, and pathogenesis of mucus hypersecretion. See Owen, Int J Chron Obstruct Pulmon Dis. 3(2):253-68 (2008); Pham et al., Nat Rev Immunol. 6(7):541-50 (2006); Fahy et al., N Engl J Med. 363(23):2233-47 (2010); Voynow et al., Am J Physiol Lung Cell Mol Physiol. 287(6):L1293-302 (2004). Hirche et al., J Immunol. 181(7):4945-54 (2008); Taggart et al., Am J Respir Crit Care Med. 171(10):1070-6 (2005); Weldon et al., J Immunol. 183(12):8148-56 (2009); each of which is incorporated herein by reference in its entirety for all purposes. These have been identified as key risk factors on the onset and progression of bronchiectasis and lung function decline in patients with CF. See Sly et al., N Engl J Med. 368(21):1963-70 (2013); Dittrich et al., Eur Respir J. 51(3) (2018); each of which is incorporated herein by reference in its entirety for all purposes. The methods provided herein employ reversible inhibitors of DPP1. Without wishing to be bound by theory, it is thought that the compounds of Formula (I), administered via the methods provided herein have beneficial effects via inhibiting the activation of NSPs and decreasing inflammation and mucus hypersecretion, which in turn leads to a decrease in pulmonary exacerbations, a decrease in the rate of pulmonary exacerbations, and/or an improvement in lung function (e.g., cough, sputum production, forced expiratory volume in 1 second [FEV₁]) in CF patients.

As used herein, “C₁₋₃” means a carbon group having 1, 2 or 3 carbon atoms.

The term “alkyl”, unless otherwise noted, includes both straight and branched chain alkyl groups and may be, substituted or non-substituted. “Alkyl” groups include, but are not limited to, methyl, ethyl, n-propyl, i-propyl, butyl, pentyl.

The term “pharmaceutically acceptable”, unless otherwise noted, is used to characterize a moiety (e.g., a salt, dosage form, or excipient) as being appropriate for use in accordance with sound medical judgment. In general, a pharmaceutically acceptable moiety has one or more benefits that outweigh any deleterious effect that the moiety may have. Deleterious effects may include, for example, excessive toxicity, irritation, allergic response, and other problems and complications.

Provided herein are methods for treating CF patients via administration of a pharmaceutical composition comprising an effective amount of a compound of Formula (I), or a pharmaceutically acceptable salt thereof, for an administration period:

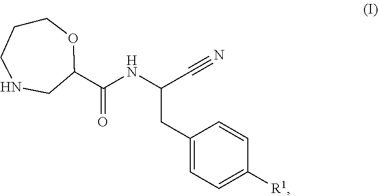

wherein,
R¹ is

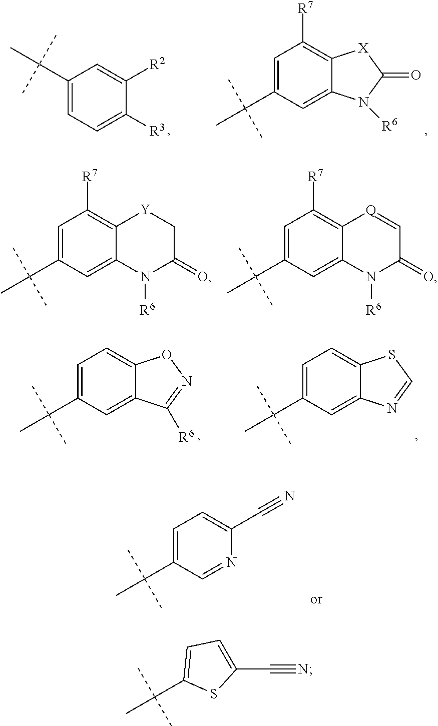

R² is hydrogen, F, Cl, Br, OSO₂C₁₋₃alkyl, or C₁₋₃alkyl;
R³ is hydrogen, F, Cl, Br, CN, CF₃, SO₂C₁₋₃alkyl, CONH₂ or SO₂NR⁴R⁵,
wherein R⁴ and R⁵ together with the nitrogen atom to which they are attached form an azetidine, pyrrolidine or piperidine ring;
X is O, S or CF₂;
Y is O or S;
Q is CH or N;
R⁶ is C₁₋₃alkyl, wherein the C₁₋₃alkyl is optionally substituted by 1, 2 or 3 F and optionally by one substituent selected from OH, OC₁₋₃alkyl, N(C₁₋₃alkyl)₂, cyclopropyl, or tetrahydropyran; and
R⁷ is hydrogen, F, Cl or CH₃;

In the methods provided herein, treating comprises (i) improving the lung function of the patient, as compared to the lung function of the patient prior to the administration period; and/or (ii) improving the patient's quality of life (QOL) assessed by the cystic fibrosis questionnaire-revised (CFQ-R), as compared to the patient's QOL assessed by the CFQ-R prior to the administration period.

In one embodiment, the compound of Formula (I) is an S,S diastereomer. In other words, the compound of Formula (I) has the following stereochemistry:

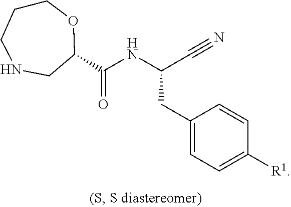

The other diastereomeric forms are also contemplated by the present invention. For example, in one embodiment, the compound of Formula (I) is the R,R diastereomer:

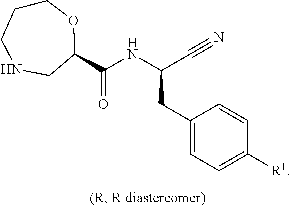

In another embodiment, the compound of Formula (I) is the R,S diastereomer:

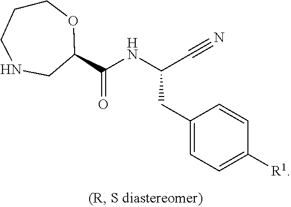

In even another embodiment, the compound of Formula (I) is the S,R diastereomer:

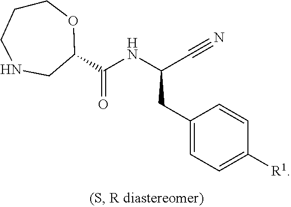

In one embodiment, the composition comprises a mixture of an S,S diastereomer of a compound of Formula (I) and an S,R diastereomer of a compound of Formula (I).

In one embodiment, the composition comprises a mixture of an S,S diastereomer of a compound of Formula (I) and an R,S diastereomer of a compound of Formula (I).

In one embodiment, the composition comprises a mixture of an S,S diastereomer of a compound of Formula (I) and an R,R diastereomer of a compound of Formula (I).

In one embodiment, R¹ is

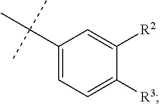

R² is hydrogen, F, Cl, Br, OSO₂C₁₋₃alkyl, or C₁₋₃alkyl; R³ is hydrogen, F, Cl, Br, CN, CF₃, SO₂C₁₋₃alkyl, CONH₂ or SO₂NR⁴R⁵, wherein R⁴ and R⁵ together with the nitrogen atom to which they are attached form an azetidine, pyrrolidine or piperidine ring. In a further embodiment, R² is hydrogen, F, Cl or C₁₋₃alkyl; and R³ is hydrogen, F, Cl, CN or SO₂C₁₋₃alkyl. In a further embodiment, R³ is hydrogen, F or CN.

In another embodiment, R¹ is

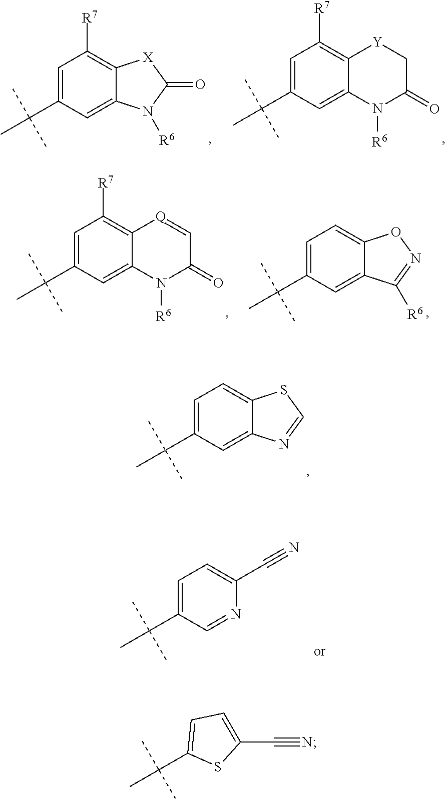

X is O, S or CF₂; Y is O or S; Q is CH or N; R⁶ is C₁₋₃alkyl, wherein the C₁₋₃alkyl is optionally substituted by 1, 2 or 3 F and optionally substituted by OH, OC₁₋₃alkyl, N(C₁₋₃alkyl)₂, cyclopropyl, or tetrahydropyran; and R⁷ is hydrogen, F, Cl or CH₃. In a further embodiment, R¹ is

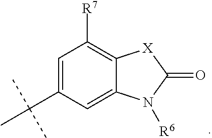

In another embodiment, R¹ is

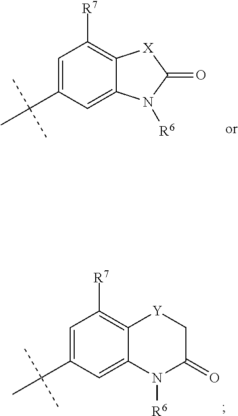

X is O, S or CF₂; Y is O or S; R⁶ is C₁₋₃alkyl, optionally substituted by 1, 2 or 3 F and optionally substituted by OH, OC₁₋₃alkyl, N(C₁₋₃alkyl)₂, cyclopropyl, or tetrahydropyran; and R⁷ is hydrogen, F, Cl or CH₃. In a further embodiment, R¹ is

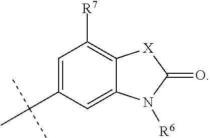

In another embodiment, R¹ is

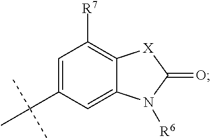

X is O, S or CF₂; R⁶ is C₁₋₃alkyl, wherein the C₁₋₃alkyl is optionally substituted by 1, 2 or 3 F; and R⁷ is hydrogen, F, Cl or CH₃.

In another embodiment, R¹ is

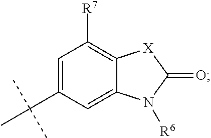

X is O; R⁶ is C₁₋₃alkyl, wherein the C₁₋₃alkyl is optionally substituted by 1, 2 or 3 F; and R⁷ is hydrogen. In a further embodiment, R⁶ is C₁₋₃alkyl, i.e., methyl, ethyl, or propyl. In still a further embodiment, R⁶ is methyl.

In one embodiment, R² is hydrogen, F, Cl, Br, OSO₂C₁₋₃alkyl or C₁₋₃alkyl.

In a further embodiment, R² is hydrogen, F, Cl or C₁₋₃alkyl.

In still a further embodiment, R² is hydrogen, F or C₁₋₃alkyl.

In one embodiment, R³ is hydrogen, F, Cl, Br, CN, CF₃, SO₂C₁₋₃alkyl CONH₂ or SO₂NR⁴R⁵, wherein R⁴ and R⁵ together with the nitrogen atom to which they are attached form an azetidine, pyrrolidine or piperidine ring.

In a further embodiment, R³ is hydrogen, F, Cl, CN or SO₂C₁₋₃alkyl.

In still a further embodiment, R³ is hydrogen, F or CN.

In one embodiment, R⁶ is C₁₋₃alkyl, wherein the C₁₋₃alkyl is optionally substituted by 1, 2 or 3 F and optionally by one substituent selected from OH, OC₁₋₃alkyl, N(C₁₋₃alkyl)₂, cyclopropyl, or tetrahydropyran.

In a further embodiment, R⁶ is C₁₋₃alkyl, wherein the C₁₋₃alkyl is optionally substituted by 1, 2 or 3 F. In still a further embodiment, R⁶ is methyl or ethyl. In still a further embodiment, R⁶ is methyl.

In one embodiment, R⁷ is hydrogen, F, Cl or CH₃. In a further embodiment R⁷ is hydrogen.

In one embodiment, the compound of Formula (I) is (2S)—N-{(1S)-1-cyano-2-[4-(3-methyl-2-oxo-2,3-dihydro-1,3-benzoxazol-5-yl)phenyl]ethyl}-1,4-oxazepane-2-carboxamide (brensocatib):

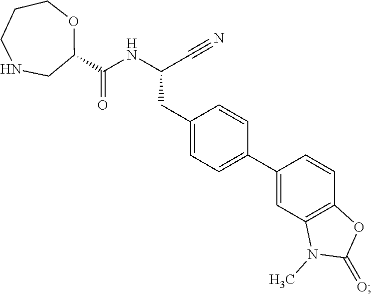 [SMILES: Cn1c(=O)oc2ccc(-c3ccc(C[C@@H](C#N)NC(=O)[C@@H]4CNCCCO4)cc3)cc21]

or a pharmaceutically acceptable salt thereof. In a further embodiment, the compound of Formula (I) is brensocatib.

In one embodiment, the compound of Formula (I) is:
(2S)—N-[(1S)-1-Cyano-2-(4′-cyanobiphenyl-4-yl)ethyl]-1,4-oxazepane-2-carboxamide,
(2S)—N-{(1S)-1-Cyano-2-[4-(3-methyl-2-oxo-2,3-dihydro-1,3-benzoxazol-5-yl)phenyl]ethyl}-1,4-oxazepane-2-carboxamide,
(2S)—N-{(1S)-1-Cyano-2-[4-(3,7-dimethyl-2-oxo-2,3-dihydro-1,3-benzoxazol-5-yl)phenyl]ethyl}-1,4-oxazepane-2-carboxamide,
4′-[(2S)-2-Cyano-2-{[(2S)-1,4-oxazepan-2-ylcarbonyl]amino}ethyl]biphenyl-3-yl methanesulfonate,
(2S)—N-{(1S)-1-Cyano-2-[4-(3-methyl-1,2-benzoxazol-5-yl)phenyl]ethyl}-1,4-oxazepane-2-carboxamide,
(2S)—N-{(1S)-1-Cyano-2-[4′-(trifluoromethyl)biphenyl-4-yl]ethyl}-1,4-oxazepane-2-carboxamide,
(2S)—N-[(1S)-1-Cyano-2-(3′,4′-difluorobiphenyl-4-yl)ethyl]-1,4-oxazepane-2-carboxamide,
(2S)—N-{(1S)-1-Cyano-2-[4-(6-cyanopyridin-3-yl)phenyl]ethyl}-1,4-oxazepane-2-carboxamide,
(2S)—N-{(1S)-1-Cyano-2-[4-(4-methyl-3-oxo-3,4-dihydro-2H-1,4-benzothiazin-6-yl)phenyl]ethyl}-1,4-oxazepane-2-carboxamide,
(2S)—N-{(1S)-1-Cyano-2-[4-(3-ethyl-7-methyl-2-oxo-2,3-dihydro-1,3-benzoxazol-5-yl)phenyl]ethyl}-1,4-oxazepane-2-carboxamide,
(2S)—N-[(1S)-1-Cyano-2-{4-[3-(2-hydroxy-2-methylpropyl)-2-oxo-2,3-dihydro-1,3-benzoxazol-5-yl]phenyl}ethyl]-1,4-oxazepane-2-carboxamide,
(2S)—N-[(1S)-1-Cyano-2-{4-[3-(2,2-difluoroethyl)-7-fluoro-2-oxo-2,3-dihydro-1,3-benzoxazol-5-yl]phenyl}ethyl]-1,4-oxazepane-2-carboxamide,
(2S)—N-[(1S)-1-Cyano-2-(4-{3-[2-(dimethylamino)ethyl]-2-oxo-2,3-dihydro-1,3-benzoxazol-5-yl}phenyl)ethyl]-1,4-oxazepane-2-carboxamide,
(2S)—N-{(1S)-1-Cyano-2-[4-(3,3-difluoro-1-methyl-2-oxo-2,3-dihydro-1H-indol-6-yl)phenyl]ethyl}-1,4-oxazepane-2-carboxamide,
(2S)—N-{(1S)-1-Cyano-2-[4-(7-fluoro-3-methyl-2-oxo-2,3-dihydro-1,3-benzoxazol-5-yl)phenyl]ethyl}-1,4-oxazepane-2-carboxamide,
(2S)—N-{(1S)-1-Cyano-2-[4-(3-ethyl-2-oxo-2,3-dihydro-1,3-benzoxazol-5-yl)phenyl]ethyl}-1,4-oxazepane-2-carboxamide,
(2S)—N-[(1S)-1-Cyano-2-{4-[3-(cyclopropylmethyl)-2-oxo-2,3-dihydro-1,3-benzoxazol-5-yl]phenyl}ethyl]-1,4-oxazepane-2-carboxamide,
(2S)—N-[(1S)-1-Cyano-2-{4-[3-(2-methoxyethyl)-2-oxo-2,3-dihydro-1,3-benzothiazol-5-yl]phenyl}ethyl]-1,4-oxazepane-2-carboxamide,
(2S)—N-[(1S)-1-Cyano-2-{4-[2-oxo-3-(propan-2-yl)-2,3-dihydro-1,3-benzoxazol-5-yl]phenyl}ethyl]-1,4-oxazepane-2-carboxamide,
(2S)—N-{(1S)-1-Cyano-2-[4-(4-methyl-3-oxo-3,4-dihydro-2H-1,4-benzoxazin-6-yl)phenyl]ethyl}-1,4-oxazepane-2-carboxamide,
(2S)—N-[(1S)-1-Cyano-2-{4-[3-(2-methoxyethyl)-2-oxo-2,3-dihydro-1,3-benzoxazol-5-yl]phenyl}ethyl]-1,4-oxazepane-2-carboxamide,
(2S)—N-{(1S)-1-Cyano-2-[4-(5-cyanothiophen-2-yl)phenyl]ethyl}-1,4-oxazepane-2-carboxamide,
(2S)—N-[(1S)-2-(4′-Carbamoyl-3′-fluorobiphenyl-4-yl)-1-cyanoethyl]-1,4-oxazepane-2-carboxamide,
(2S)—N-{(1S)-1-Cyano-2-[4-(1-methyl-2-oxo-1,2-dihydroquinolin-7-yl)phenyl]ethyl}-1,4-oxazepane-2-carboxamide,
(2S)—N-[(1S)-1-Cyano-2-{4-[2-oxo-3-(tetrahydro-2H-pyran-4-ylmethyl)-2,3-dihydro-1,3-benzoxazol-5-yl]phenyl}ethyl]-1,4-oxazepane-2-carboxamide,
(2S)—N-{(1S)-2-[4-(7-Chloro-3-methyl-2-oxo-2,3-dihydro-1,3-benzoxazol-5-yl)phenyl]-1-cyanoethyl}-1,4-oxazepane-2-carboxamide,
(2S)—N-[(1S)-1-Cyano-2-{4-[3-(2,2-difluoroethyl)-2-oxo-2,3-dihydro-1,3-benzoxazol-5-yl]phenyl}ethyl]-1,4-oxazepane-2-carboxamide,
(2S)—N-[(1S)-1-Cyano-2-{4-[2-oxo-3-(2,2,2-trifluoroethyl)-2,3-dihydro-1,3-benzoxazol-5-yl]phenyl}ethyl]-1,4-oxazepane-2-carboxamide,
(2S)—N-{(1S)-1-Cyano-2-[4-(3-methyl-2-oxo-2,3-dihydro-1,3-benzothiazol-5-yl)phenyl]ethyl}-1,4-oxazepane-2-carboxamide,
(2S)—N-{(1S)-1-Cyano-2-[4′-(methylsulfonyl)biphenyl-4-yl]ethyl}-1,4-oxazepane-2-carboxamide,
(2S)—N-{(1S)-2-[4′-(Azetidin-1-ylsulfonyl)biphenyl-4-yl]-1-cyanoethyl}-1,4-oxazepane-2-carboxamide,
(2S)—N-[(1S)-1-Cyano-2-(4′-fluorobiphenyl-4-yl)ethyl]-1,4-oxazepane-2-carboxamide,
(2S)—N-{(1S)-2-[4-(1,3-Benzothiazol-5-yl)phenyl]-1-cyanoethyl}-1,4-oxazepane-2-carboxamide, or
(2S)—N-[(1S)-1-Cyano-2-(4′-cyanobiphenyl-4-yl)ethyl]-1,4-oxazepane-2-carboxamide,
   or a pharmaceutically acceptable salt of one of the foregoing compounds.

In one embodiment, the compound of Formula (I) is brensocatib. In some embodiments, brensocatib is in polymorphic Form A as disclosed in U.S. Pat. No. 9,522,894, the disclosure of which is incorporated herein by reference in its entirety for all purposes. In some embodiments, brensocatib is characterized by an X-ray powder diffraction pattern having a peak at about 12.2±0.2 (° 2-theta), measured using CuKα radiation. In some embodiments, brensocatib is characterized by an X-ray powder diffraction pattern having a peak at about 20.6±0.2 (° 2-theta), measured using CuKα radiation. In some embodiments, brensocatib is characterized by an X-ray powder diffraction pattern having a peak at about 12.2±0.2 and about 20.6±0.2 (° 2-theta), measured using CuKα radiation. In some embodiments, brensocatib is characterized by an X-ray powder diffraction pattern having a peak at about 12.2±0.2, about 14.3±0.2, about 16.2±0.2, about 19.1±0.2 and about 20.6±0.2 (° 2-theta), measured using CuKα radiation.

As provided throughout, according to the methods provided herein, a compound of Formula (I) can be administered as a pharmaceutically acceptable salt. A pharmaceutically acceptable salt of a compound of Formula (I) may be advantageous due to one or more of its chemical or physical properties, such as stability in differing temperatures and humidities, or a desirable solubility in H₂O, oil, or other solvent. In some instances, a salt may be used to aid in the isolation or purification of the compound of Formula (I).

Where the compound of Formula (I) is sufficiently acidic, pharmaceutically acceptable salts include, but are not limited to, an alkali metal salt, e.g., Na or K, an alkali earth metal salt, e.g., Ca or Mg, or an organic amine salt. Where the compound of Formula (I) is sufficiently basic, pharmaceutically acceptable salts include, but are not limited to, inorganic or organic acid addition salts.

There may be more than one cation or anion depending on the number of charged functions and the valency of the cations or anions.

For reviews on suitable salts, and pharmaceutically acceptable salts amenable for use herein, see Berge et al., J. Pharm. Sci., 1977, 66, 1-19 or “Handbook of Pharmaceutical Salts: Properties, selection and use”, P. H. Stahl, P. G. Vermuth, IUPAC, Wiley-VCH, 2002, incorporated by reference herein in its entirety for all purposes.

The compounds of Formula (I) may form mixtures of its salt and co-crystal forms. It is also to be understood that the methods provided herein can employ such salt/co-crystal mixtures of the compound of Formula (I).

Salts and co-crystals may be characterized using well known techniques, for example X-ray powder diffraction, single crystal X-ray diffraction (for example to evaluate proton position, bond lengths or bond angles), solid state NMR, (to evaluate for example, C, N or P chemical shifts) or spectroscopic techniques (to measure for example, O—H, N—H or COOH signals and IR peak shifts resulting from hydrogen bonding).

It is also to be understood that compounds of Formula (I) may exist in solvated form, e.g., hydrates, including solvates of a pharmaceutically acceptable salt of a compound of Formula (I).

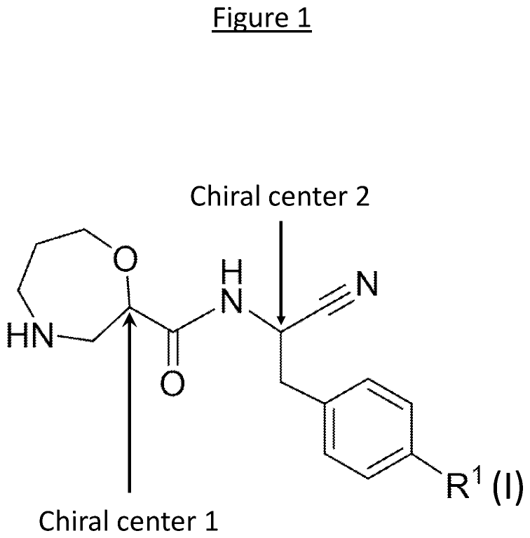

In one embodiment, compounds of Formula (I) may exist as racemates and racemic mixtures, single enantiomers, individual diastereomers and diastereomeric mixtures. It is to be understood that the present disclosure encompasses all such isomeric forms, even though the compound of Formula (I), in its preferred form, has S,S stereochemistry. As shown in FIG. 1, irrespective of R¹, the backbone of the compounds of Formula (I) has two chiral centers. Chiral center 1 is the most substituted carbon atom on the 1,4-oxazepane ring. Chiral center 2 is the substituted carbon atom to which a cyano group, —NH—, and a benzyl group are attached. The present disclosure encompasses the compounds of Formula (I) with the (S)-configuration for the ring substituent at chiral center 1 and the (S)-configuration for the benzyl substituent at chiral center 2 (i.e., the S,S diastereomer disclosed herein); the (S)-configuration for the ring substituent at chiral center 1 and the (R)-configuration for the benzyl substituent at chiral center 2 (i.e., the S,R diastereomer disclosed herein); the (R)-configuration for the ring substituent at chiral center 1 and the (S)-configuration for the benzyl substituent at chiral center 2 (i.e., the R,S diastereomer disclosed herein); and the (R)-configuration for the ring substituent at chiral center 1 and the (R)-configuration for the benzyl substituent at chiral center 2 (i.e., the R,R diastereomer disclosed herein), as well as a mixture of any two or more of the foregoing diastereomers.

Accordingly, in one embodiment, the compound of Formula (I) is (2S)—N-{(1S)-1-Cyano-2-[4-(3-methyl-2-oxo-2,3-dihydro-1,3-benzoxazol-5-yl)phenyl]ethyl}-1,4-oxazepane-2-carboxamide (i.e., brensocatib, the S,S isomer), shown below.

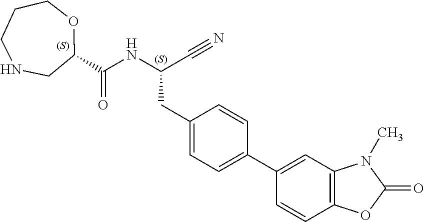

or a pharmaceutically acceptable salt thereof.

In one embodiment, the compound of Formula (I) is (2R)—N-{(1R)-1-Cyano-2-[4-(3-methyl-2-oxo-2,3-dihydro-1,3-benzoxazol-5-yl)phenyl]ethyl}-1,4-oxazepane-2-carboxamide (i.e., the R,R isomer), shown below.

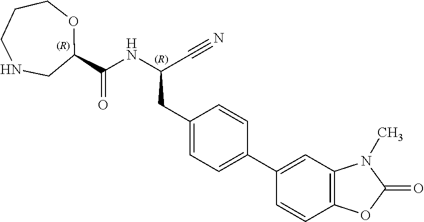

or a pharmaceutically acceptable salt thereof.

In one embodiment, the compound of Formula (I) is (2S)—N-{(1R)-1-Cyano-2-[4-(3-methyl-2-oxo-2,3-dihydro-1,3-benzoxazol-5-yl)phenyl]ethyl}-1,4-oxazepane-2-carboxamide (i.e., the S,R isomer), shown below.

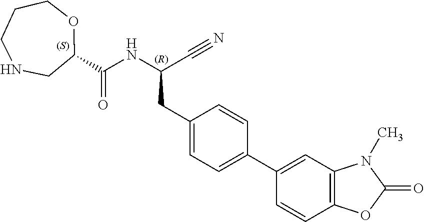

or a pharmaceutically acceptable salt thereof.

In one embodiment, the compound of Formula (I) is (2R)—N-{(1S)-1-Cyano-2-[4-(3-methyl-2-oxo-2,3-dihydro-1,3-benzoxazol-5-yl)phenyl]ethyl}-1,4-oxazepane-2-carboxamide (i.e., the R,S isomer), shown below.

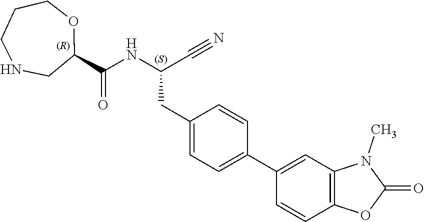

or a pharmaceutically acceptable salt thereof.

In one embodiment, the composition comprises a mixture of two or more of the aforementioned stereoisomers. The mixture in one embodiment, comprises the S,S isomer (brensocatib) and the S,R isomer of a compound of Formula (I). In another embodiment, the composition comprises a mixture of the S,S isomer (brensocatib) and the R,S isomer. In yet another embodiment, the composition comprises a mixture of the S,S isomer (brensocatib) and the R,R isomer.

Certain compounds of Formula (I) may also contain linkages (e.g. carbon-carbon bonds, carbon-nitrogen bonds such as amide bonds) wherein bond rotation is restricted about that particular linkage, e.g. restriction resulting from the presence of a ring bond or double bond. Accordingly, it is to be understood that the present disclosure encompasses all such isomers. Certain compounds of Formula (I) may also contain multiple tautomeric forms. It is to be understood that the present disclosure encompasses all such tautomeric forms. Stereoisomers may be separated using conventional techniques, e.g. chromatography or fractional crystallization, or the stereoisomers may be made by stereoselective synthesis.

In a further embodiment, the compounds of Formula (I) encompass any isotopically-labeled (or “radio-labelled”) derivatives of a compound of Formula (I). Such a derivative is a derivative of a compound of Formula (I) wherein one or more atoms are replaced by an atom having an atomic mass or mass number different from the atomic mass or mass number typically found in nature. Examples of radionuclides that may be incorporated include ²H (also written as “D” for deuterium). As such, in one embodiment, a compound of Formula (I) is provided where one or more hydrogen atoms are replaced by one or more deuterium atoms; and the deuterated compound is used in one of the methods provided herein for treating CF.

In a further embodiment, the compounds of Formula (I) may be administered in the form of a prodrug which is broken down in the human or animal body to give a compound of the Formula (I). Examples of prodrugs include in vivo hydrolysable esters of a compound of the Formula (I).

An in vivo hydrolysable (or cleavable) ester of a compound of Formula (I) that contains a carboxy or a hydroxy group is, for example, a pharmaceutically acceptable ester which is hydrolyzed in the human or animal body to produce the parent acid or alcohol. For examples of ester prodrugs derivatives, see: Curr. Drug. Metab. 2003, 4, 461, incorporated by reference herein in its entirety for all purposes.

Various other forms of prodrugs are known in the art, and can be used in the methods provided herein. For examples of prodrug derivatives, see: Nature Reviews Drug Discovery 2008, 7, 255, the disclosure of which is incorporated by reference herein in its entirety for all purposes.

The methods provided herein comprise the administration of a composition comprising an effective amount of a compound of Formula (I), or a pharmaceutically acceptable salt thereof, to a CF patient in need of treatment. The compounds of Formula (I) and their pharmaceutically acceptable salts are reversible inhibitors of dipeptidyl peptidase 1 (DPP1) activity. Administration routes include oral administration. Administration schedules and administration periods can be determined by the user of the method, e.g., a prescribing physician. In one embodiment, administration is once daily. In another embodiment, administration is twice daily. In another embodiment, administration is every other day, every third day, 3× per week or 4× per week.

In one embodiment, a method for treating CF is provided comprising administering to a patient in need thereof, a composition comprising an effective amount of a compound of Formula (I), or a pharmaceutically acceptable salt thereof. In one embodiment, the compound is administered orally, once daily. The method comprises improving the lung function of the patient, as compared to the lung function of the patient prior to the administration period. In a further embodiment, the compound of Formula (I) is brensocatib, or a pharmaceutically acceptable salt thereof.

The improvement in lung function in one embodiment, is measured by spirometry.

Improving the lung function of the patient, in one embodiment, comprises increasing the patient's forced expiratory volume in 1 second (FEV₁), increasing the patient's percentage of the predicted FEV₁ (ppFEV₁), increasing the patient's forced vital capacity (FVC), increasing the patient's peak expiratory flow rate (PEFR), and/or increasing the patient's forced expiratory flow between 25% and 75% of FVC (FEF_{(25-75%)}), as compared to the respective value prior to the administration period. Increasing, in one embodiment, is by about 5%, by about 10%, by about 15%, by about 20%, by about 25%, by about 30%, by about 35%, by about 40%, by about 45% or by about 50% of the respective value. Increasing, in one embodiment, is by at least about 5%, by at least about 10%, by at least about 15%, by at least about 20%, by at least about 25%, by at least about 30%, by at least about 35%, by at least about 40%, by at least about 45% or by at least about 50%. In yet another embodiment, the increasing is by about 5% to about 50%, by about 5% to about 40%, by about 5% to about 30% or by about 5% to about 20%. In even another embodiment, increasing is by about 10% to about 50%, by about 15% to about 50%, by about 20% to about 50%, or by about 25% to about 50%.

The assessment of lung function, e.g., via FEV₁, ppFEV₁, FVC, PEFR or FEF_{(25-75%)} measurement, in one embodiment, comprises comparing the lung function in the patient prior to the administration period, e.g., immediately prior to treatment, to a time point during the administration period, to an average of measurements taken during the administration period, or subsequent to the administration period.

As provided herein, treatment via a method of the invention, in one embodiment, comprises improving the lung function in the patient, wherein the lung function is measured by spirometry. Spirometry is a physiological test that measures how an individual inhales or exhales volumes of air. The primary signal measured in spirometry may be volume or flow. For the methods described herein, pulmonary function test (PFT) by spirometry (e.g., FEV₁, FVC, PEFR, and FEF_{(25-75%)} is performed per the American Thorasic Society (ATS)/European Respiratory Society (ERS) criteria, e.g., as set forth by Miller et al. (Miller et al. (2005). Standardization of Spirometry. Eur. Respir. J. 26, pp. 319-38, incorporated by reference herein in its entirety for all purposes).

In one embodiment, the spirometer is capable of accumulating volume for greater than or equal to 15 seconds, e.g., ≥20 seconds, ≥25 seconds, ≥30 seconds, ≥35 seconds. The spirometer in one embodiment can measure volumes of ≥8 L (BTPS) with an accuracy of at least ±3% of reading or ±0.050 L, whichever is greater, with flows between 0 and 14 L·s⁻¹. In one embodiment, the total resistance to airflow of the spirometer at 14 L·s⁻¹ is <1.5 cmH₂O·L⁻¹s⁻¹ (0.15 kPa? L⁻¹·s⁻¹). In one embodiment, the total resistance of the spirometer is measured with any tubing, valves, pre-filter, etc. included that may be inserted between the patient and the spirometer. With respect to devices that exhibit changes in resistance due to water vapor condensation, in one embodiment, spirometer accuracy requirements are met under BTPS (body temperature, ambient pressure, saturated with water vapor) conditions for up to eight successive FVC maneuvers performed in a 10-min period without inspiration from the instrument.

With respect to the forced expiratory maneuvers described herein, in one embodiment, the range and accuracy recommendations as set forth in Table 6 of Miller et al. are met (Miller et al. (2005). Standardization of Spirometry. Eur. Respir. J. 26, pp. 319-38, incorporated by reference herein in its entirety for all purposes).

In one embodiment, the improvement in lung function is an improvement in the forced vital capacity (FVC), i.e., the maximal volume of air exhaled with maximally forced effort from a maximal inspiration. This measurement is expressed in liters at body temperature and ambient pressure saturated with water vapor (BTPS).

“Forced vital capacity” (FVC) denotes the volume of gas which is exhaled during a forced expiration starting from a position of full inspiration and ending at complete expiration and is one measure of treatment efficacy. In one embodiment of the methods provided herein, improving the patient's lung function comprises increasing the patient's FVC, compared to the patient's FVC prior to the administration period. In one embodiment, the FVC of a treated patient is greater by about 1%, greater by about 2%, greater by about 3%, greater by about 4%, greater by about 5%, greater by about 6%, greater by about 7%, greater by about 8%, greater by about 9%, greater by about 10%, greater by about 110%, greater by about 12%, greater by about 13%, greater by about 14%, greater by about 15%, greater by about 16%, greater by about 17%, greater by about 18%, greater by about 19%, greater by about 20%, greater by about 25%, greater by about 30%, greater by about 35%, greater by about 40%, greater by about 45%, greater by about 50%, greater by about 55%, greater by about 60%, greater by about 65%, greater by about 70%, greater by about 75%, greater by about 80%, greater by about 85% or greater by about 90%, as compared to the patient's FVC prior to the administration period. In another embodiment, the FVC of a treated patient is greater by at least about 5%, by at least about 10%, by at least about 15%, by at least about 20%, by at least about 25%, by at least about 30%, by at least about 35%, by at least about 40%, by at least about 45%, or by at least about 50%, as compared to the patient's FVC prior to the administration period. In another embodiment, the FVC of a treated patient is greater by about 5% to about 50%, by about 5% to about 40%, by about 5% to about 30%, by about 5% to about 20%, by about 10% to about 50%, by about 15% to about 50%, by about 20% to about 50%, or by about 25% to about 50%, as compared to the patient's FVC prior to the administration period. In one embodiment, the increase in FVC is an increase in pre-bronchodilator FVC. In another embodiment, the increase in FVC is an increase in post-bronchodilator FVC.

FVC maneuvers can be performed according to the procedures known to those of ordinary skill in the art. Briefly, the three distinct phases to the FVC maneuver are (1) maximal inspiration; (2) a “blast” of exhalation and (3) continued complete exhalation to the end of test (EOT). The maneuver can be carried out via the closed circuit method or open circuit method. In either instance, the subject inhales rapidly and completely with a pause of less than 1 second at total lung capacity (TLC). The subject then exhales maximally until no more air can be expelled while maintaining an upright posture. The exhalation begins with a “blast” of air from the lungs and then is encouraged to fully exhale. Enthusiastic coaching of the subject continues for a minimum of three maneuvers.

The improvement in lung function, in one embodiment, is an improvement compared to lung function immediately prior to the administration period. FEV is the volume of gas exhaled in a specified time (typically 1 second, i.e., FEV₁) from the start of the forced vital capacity maneuver (Quanjer et al. (1993). Eur. Respir. J. 6, Suppl. 16, pp. 5-40, incorporated by reference herein in its entirety for all purposes). FEV₁ may also be expressed as a percentage of the predicted FEV₁ (i.e., ppFEV₁) obtained from a normal population, based on the patient's gender, height, and age, and sometimes race and weight.

The increase in FEV₁, in one embodiment, is an increase of at least about 5%, for example, about 5% to about 50%, about 10% to about 50%, or about 15% to about 50%. In another embodiment, the FEV₁ of the treated patient is greater by about 1%, greater by about 2%, greater by about 3%, greater by about 4%, greater by about 5%, greater by about 6%, greater by about 7%, greater by about 8%, greater by about 9%, greater by about 10%, greater by about 11%, greater by about 12%, greater by about 13%, greater by about 14%, greater by about 15%, greater by about 16%, greater by about 17%, greater by about 18%, greater by about 19%, greater by about 20%, greater by about 25%, greater by about 30%, greater by about 35%, greater by about 40%, greater by about 45%, greater by about 50%, greater by about 55%, greater by about 60%, greater by about 65%, greater by about 70%, greater by about 75%, greater by about 80%, greater by about 85%, or greater by about 90%, compared to the patient's FEV₁ prior to the administration period. In one embodiment, the increase in FEV₁ is an increase in pre-bronchodilator FEV₁. In another embodiment, the increase in FEV₁ is an increase in post-bronchodilator FEV₁.

In another embodiment, improving the lung function of the patient comprises increasing the patient's FEV₁ about 25 mL to about 500 mL, or about 25 mL to about 250 mL, or about 50 mL to about 200 mL, as compared to the patient's FEV₁ prior to the administration period. In one embodiment, the increase in FEV₁ is an increase in pre-bronchodilator FEV₁. In another embodiment, the increase in FEV₁ is an increase in post-bronchodilator FEV₁.

In one embodiment, improving the lung function of the patient comprises increasing the patient's ppFEV₁ compared to the patient's ppFEV₁ prior to the administration period. The increase in ppFEV₁, in one embodiment, is an increase of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 85%, or about 90%. In another embodiment, the increase in ppFEV₁ is an increase of about 5%, about 10%, about 15%, about 20%, about 25%, about 30%, about 35%, about 40%, about 45%, or about 50%. In another embodiment, the increase in ppFEV₁ is an increase of at least about 5%, at least about 10%, at least about 15%, at least about 20%, at least about 25%, at least about 30%, at least about 35%, at least about 40%, at least about 45%, or at least about 50%. In another embodiment, the increase in ppFEV₁ is an increase of about 5% to about 50%, about 5% to about 40%, about 5% to about 30%, about 5% to about 20%, about 10% to about 50%, about 15% to about 50%, about 20% to about 50%, or about 25% to about 50%.

In one embodiment, the increase in ppFEV₁ is an increase in pre-bronchodilator ppFEV₁. In another embodiment, the increase in ppFEV₁ is an increase in post-bronchodilator ppFEV₁.

In one embodiment, the patient's ppFEV₁ is about 40% or greater prior to the administration period. In another embodiment, the patient's ppFEV₁ is about 50% or greater prior to the administration period. In another embodiment, the patient's ppFEV₁ is about 60% or greater prior to the administration period. In another embodiment, the patient's ppFEV₁ is about 70% or greater prior to the administration period. In one embodiment, the patient's ppFEV₁ is from about 40% to about 90% prior to the administration period. In another embodiment, the patient's ppFEV₁ is from about 40% to about 80% prior to the administration period. In another embodiment, the patient's ppFEV₁ is from about 50% to about 80% prior to the administration period. In another embodiment, the patient's ppFEV₁ is from about 50% to about 70% prior to the administration period.

Oxygen saturation is an indication of how much hemoglobin in the blood is bound to oxygen, and is typically provided as a percentage of oxyhemoglobin to the total hemoglobin. Saturation of peripheral capillary oxygenation (SpO₂) is an indication of oxygen saturation in the peripheral capillaries. Exemplary methods to measure SpO₂ include, but are not limited to, pulse oximetry using a pulse oximeter. In one embodiment, the patient's SpO₂ on room air is greater than about 90% prior to the administration period. In another embodiment, the patient's SpO₂ on room air is greater than about 92% prior to the administration period. In another embodiment, the patient's SpO₂ on room air is greater than about 95% prior to the administration period.

In one embodiment, improving the lung function of the patient comprises increasing the mean forced expiratory flow between 25% and 75% of FVC (FEF_{(25-75%)}) (also referred to as the maximum mid-expiratory flow) of the patient, as compared to the patient's FEF_{(25-75%)} prior to the administration period. The increase in FEF_{(25-75%)}, in one embodiment, is an increase of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 85%, or about 90%. In another embodiment, the increase in FEF_{(25-75%)} is an increase of about 5%, about 10%, about 15%, about 20%, about 25%, about 30%, about 35%, about 40%, about 45%, or about 50%. In another embodiment, the increase in FEF_{(25-75%)} is an increase of at least about 5%, at least about 10%, at least about 15%, at least about 20%, at least about 25%, at least about 30%, at least about 35%, at least about 40%, at least about 45%, or at least about 50%. In another embodiment, the increase in FEF_{(25-75%)} is an increase of about 5% to about 50%, about 5% to about 40%, about 5% to about 30%, about 5% to about 20%, about 10% to about 50%, about 15% to about 50%, about 20% to about 50%, or about 25% to about 50%. In one embodiment, the increase in FEF_{(25-75%)} is an increase in pre-bronchodilator FEF_{(25-75%)}. In another embodiment, the increase in FEF_{(25-75%)} is an increase in post-bronchodilator FEF_{(25-75%)}. The measurement is dependent on the validity of the FVC measurement and the level of expiratory effort. The FEF_{(25-75%)} index is taken from the blow with the largest sum of FEV₁ and FVC.

In one embodiment, improving the lung function of the patient comprises increasing the peak expiratory flow rate (PEFR) of the patient. In one embodiment, the increasing is an increase compared to the patient's PEFR immediately prior to the administration period. The PEFR measures the fastest rate of air that can be expired by a subject. In one embodiment, the PEFR of a treated patient is greater by about 1%, greater by about 2%, greater by about 3%, greater by about 4%, greater by about 5%, greater by about 6%, greater by about 7%, greater by about 8%, greater by about 9%, greater by about 10%, greater by about 11%, greater by about 12%, greater by about 13%, greater by about 14%, greater by about 15%, greater by about 16%, greater by about 17%, greater by about 18%, greater by about 19%, greater by about 20%, greater by about 25%, greater by about 30%, greater by about 35%, greater by about 40%, greater by about 45%, greater by about 50%, greater by about 55%, greater by about 60%, greater by about 65%, greater by about 70%, greater by about 75%, greater by about 80%, greater by about 85%, or greater by about 90%. In another embodiment, the PEFR of a treated patient is greater by at least about 5%, by at least about 10%, by at least about 15%, by at least about 20%, by at least about 25%, by at least about 30%, by at least about 35%, by at least about 40%, by at least about 45%, or by at least about 50%. In another embodiment, the PEFR of a treated patient is greater by about 5% to about 50%, by about 5% to about 40%, by about 5% to about 30%, by about 5% to about 20%, by about 10% to about 50%, by about 15% to about 50%, by about 20% to about 50%, or by about 25% to about 50%. In one embodiment, the increase in PEFR is an increase in pre-bronchodilator PEFR. In another embodiment, the increase in PEFR is an increase in post-bronchodilator PEFR.

In yet another embodiment of the invention, a method for treating CF is provided comprising administering a composition comprising an effective amount of a compound of Formula (I) to a patient in need thereof, wherein treating comprises improving the quality of life (QOL) of the patient assessed by the cystic fibrosis questionnaire-revised (CFQ-R), as compared to the quality of life of the patient prior to the administration period, e.g., a baseline value. In a further embodiment, the QOL is assessed by a respiratory domain score of the CFQ-R. In one embodiment, the compound is administered orally, once daily. The compound of Formula (I) in one embodiment, is brensocatib, or a pharmaceutically acceptable salt thereof. The Cystic Fibrosis Questionnaire-Revised (CFQ-R) is a disease-specific validated instrument for assessing health-related quality of life (HRQOL) in children, adolescents and adults with cystic fibrosis (CF). It is a profile measure of HRQOL with 9 QOL domains (Physical Functioning, Vitality, Emotional State, Social Limitations, Role Limitations/School Performance, Embarrassment, Body Image, Eating Disturbances, Treatment Constraints) that assess the impact of CF on overall health, daily life, and perceived well-being. There are 3 symptom scales: weight, respiratory, and digestion. Scores range from 0 to 100, with higher scores indicating better health. See Henry et al., “Development of the Cystic Fibrosis Questionnaire (CFQ) for assessing quality of life in pediatric and adult patients,” Qual Life Res. 12(1):63-76 (2003); Quittner et al., “Determination of the minimal clinically important difference scores for the Cystic Fibrosis Questionnaire-Revised respiratory symptom scale in two populations of patients with cystic fibrosis and chronic Pseudomonas aeruginosa airway infection,” Chest. 135(6):1610-1618 (2009), each of which is incorporated by reference herein in its entirety for all purposes.

In another embodiment of the method for treating CF provided herein, a composition comprising an effective amount of a compound of Formula (I), or a pharmaceutically acceptable salt thereof, is administered to a patient in need thereof, wherein treating further comprises decreasing a sputum concentration of an active NSP in the patient, as compared to the patient's active NSP sputum concentration, prior to the administration period. In one embodiment, the compound of Formula (I) is administered via oral administration. In a further embodiment, administration is 1× daily, every other day, 2× weekly, 3× weekly or 4× weekly. In a further embodiment, administration is 1× daily. The compound of Formula (I) in one embodiment, is brensocatib, or a pharmaceutically acceptable salt thereof. In one embodiment, the active NSP is active NE. In another embodiment, the active NSP is active PR3. In another embodiment, the active NSP is active CatG.

Decreasing the patient's active NSP sputum concentration, in one embodiment, comprises decreasing by about 1%, about 5%, about 10%, about 20%, about 25%, about 30%, about 40%, about 50%, about 60%, about 70%, or about 80%. In another embodiment, decreasing the patient's active NSP sputum concentration comprises decreasing by at least about 1%, at least about 5%, at least about 10%, at least about 20%, at least about 30%, at least about 40%, at least about 50%, at least about 60%, at least about 70%, or at least about 80%. In one embodiment, the active NSP is active NE. In another embodiment, the active NSP is active PR3. In another embodiment, the active NSP is active CatG.

In another embodiment of the method for treating CF provided herein, a composition comprising an effective amount of a compound of Formula (I), or a pharmaceutically acceptable salt thereof, is administered to a patient in need thereof, wherein treating further comprises decreasing a concentration of an active NSP in the blood of the patient, as compared to the patient's active NSP blood concentration, prior to the administration period. In one embodiment, the compound of Formula (I) is administered via oral administration. In a further embodiment, administration is 1× daily, every other day, 2× weekly, 3× weekly or 4× weekly. In a further embodiment, administration is 1× daily. The compound of Formula (I) in one embodiment, is brensocatib, or a pharmaceutically acceptable salt thereof. In one embodiment, the active NSP is active NE. In another embodiment, the active NSP is active PR3. In another embodiment, the active NSP is active CatG.

Decreasing the patient's active NSP blood concentration, in one embodiment, comprises decreasing by about 1%, about 5%, about 10%, about 20%, about 25%, about 30%, about 40%, about 50%, about 60%, about 70%, or about 80%. In another embodiment, decreasing the patient's active NSP blood concentration comprises decreasing by at least about 1%, at least about 5%, at least about 10%, at least about 20%, at least about 30%, at least about 40%, at least about 50%, at least about 60%, at least about 70%, or at least about 80%. In one embodiment, the active NSP is active NE. In another embodiment, the active NSP is active PR3. In another embodiment, the active NSP is active CatG.

CF patients likely develop bacterial infections in the lungs due to the buildup of thick, sticky mucus in the lungs. Bacteria causing the infections include, but are not limited to, Achromobacter xylosoxidans, Burkholderia cepacian, Haemophilus influenzae, nontuberculous mycobacteria (NTM) (e.g., Mycobacterium abscessus, and Mycobacterium avium complex (MAC)), Pseudomonas (e.g., P. aeruginosa), Staphylococcus aureus, methicillin-resistant Staphylococcus aureus (MRSA), and Stenotrophomonas maltophilia. In one embodiment of the invention, a method for treating CF is provided comprising administering a composition comprising an effective amount of a compound of Formula (I) to a patient in need thereof, wherein treating further comprises decreasing a bacterial infection in the lung of the patient, as compared to the bacterial infection in the lung of the patient prior to the administration period. In one embodiment, the compound is administered orally, once daily. The compound of Formula (I) in one embodiment, is brensocatib, or a pharmaceutically acceptable salt thereof. In one embodiment, the bacterial infection comprises a Pseudomonas infection, e.g., Pseudomonas aeruginosa infection. In another embodiment, the bacterial infection comprises Staphylococcus aureus infection. In a further embodiment, the Staphylococcus aureus infection is a methicillin-resistant Staphylococcus aureus (MRSA) infection. In another embodiment, the bacterial infection comprises Haemophilus influenzae infection. In another embodiment, the bacterial infection comprises Stenotrophomonas maltophilia infection. In another embodiment, the bacterial infection comprises Burkholderia cepacia complex infection. In a further embodiment, the bacterial infection comprises Burkholderia cenocepacia infection.

In one embodiment, decreasing the bacterial infection in the lung of the patient comprises decreasing a number of colony forming units (CFUs) of the bacteria present in the patient's sputum, as compared to a number of CFUs of the bacteria present in the patient's sputum prior to the administration period. In one embodiment, the number of CFUs of the bacteria present in the treated patient's sputum is decreased about 1%, about 5%, about 10%, about 20%, about 25%, about 30%, about 40%, about 50%, about 60%, about 70%, about 80%, or about 90%. In another embodiment, the number of CFUs of the bacteria present in the treated patient's sputum is decreased at least about 1%, at least about 5%, at least about 10%, at least about 20%, at least about 30%, at least about 40%, at least about 50%, at least about 60%, at least about 70%, at least about 80%, or at least about 90%. In another embodiment, the number of CFUs of the bacteria present in the treated patient's sputum is decreased by about 5% to about 50%, by about 5% to about 40%, by about 5% to about 30%, by about 5% to about 20%, by about 10% to about 50%, by about 15% to about 50%, by about 20% to about 50%, or by about 25% to about 50%.

In another embodiment, the methods provided herein comprise co-therapy with an antibiotic. In one embodiment, the patient administered the antibiotic in combination with a compound of Formula (I) or its pharmaceutically acceptable salt has not previously been treated with the antibiotic. Alternatively, the patient may have previously been treated with the antibiotic. The antibiotic may be adminstered to the patient via oral administration, intravenous administration, intramuscular administration, topical administration, or inhalation. In some embodiments, the antibiotic is selected from the group consisting of an aminoglycoside, aztreonam, a carbapenem, a cephalosporin, clofazimine, colistimethate, ethambutol, a lincosamide, a macrolide, an oxazolidinone, a penicillin, a quinolone, a rifamycin, a sulfa, a tetracycline, vancomycin, and a combination thereof. In one embodiment, the antibiotic is selected from the group consisting of amikacin, aztreonam, colistimethate, gentamicin, tobramycin, or a combination thereof. In a further embodiment, amikacin (Arikayce®), aztreonam, colistimethate (Colistin®), gentamicin, tobramycin, or a combination thereof is administered to the patient via inhalation. The antibiotic may be administered concurrently, sequentially or in admixture with a compound of Formula (I) or its pharmaceutically acceptable salt.

In one embodiment of the methods, the patient has previously been treated with a cystic fibrosis transmembrane conductance regulator (CFTR) modulator.

In one embodiment, the methods provided herein comprise co-therapy with a CFTR modulator. The patient administered co-therapy may be CFTR modulator treatment naïve, i.e., may never have received the CFTR modulator. Alternatively, the patient may have previously been treated with a CFTR modulator. CFTR modulators target the defects in CFTR caused by certain mutations in the CFTR gene. In a further embodiment, the CFTR modulator is ivacaftor, lumacaftor, tezacaftor, elexacaftor, or a combination thereof. The CFTR modulator may be administered concurrently, sequentially or in admixture with a compound of Formula (I) or its pharmaceutically acceptable salt.

In one embodiment of the methods, the patient has not previously been treated with a CFTR modulator. In a further embodiment, the CFTR treatment naïve patient is not administered a CFTR modulator.

The dosage administered will vary with the compound of Formula (I) employed, the mode of administration, and the treatment outcome desired. For example, in one embodiment, the daily dosage of the compound of Formula (I), if inhaled, may be in the range from 0.05 micrograms per kilogram body weight (μg/kg) to 100 micrograms per kilogram body weight (μg/kg). Alternatively, in one embodiment, if the compound of Formula (I) is administered orally, then the daily dosage of the compound of the disclosure may be in the range from 0.01 micrograms per kilogram body weight (μg/kg) to 100 milligrams per kilogram body weight (mg/kg). In another embodiment, the daily dosage of the compound of Formula (I) is from about 5 mg to about 70 mg, from about 10 mg to about 40 mg, about 10 mg, about 25 mg, about 40 mg, or about 65 mg. In a further embodiment, the compound of Formula (I) is administered orally. In a further embodiment, the compound of Formula (I) is brensocatib, or a pharmaceutically acceptable salt thereof.

In one embodiment, the compound of Formula (I) is administered in an oral dosage form. In a further embodiment, the compound of Formula (I) is administered as a 5 mg to 70 mg, or 10 mg to 40 mg dosage form, for example, a 10 mg dosage form, a 15 mg dosage form, a 20 mg dosage form, a 25 mg dosage form, a 30 mg dosage form, a 40 mg dosage form, or a 65 mg dosage form. In a further embodiment, the dosage form is 10 mg, 25 mg, or 40 mg or 65 mg. In a further embodiment, the dosage form is administered once daily. In even a further embodiment, the compound is brensocatib, or a pharmaceutically acceptable salt thereof.

Treating, in one embodiment, is carried out over an administration period of at least 1 month, from about 1 month to about 12 months, e.g., about 1, 2, 3, 4, 5, 6, 7, 8, 9, 10, 11, or 12 months, from about 6 months to about 24 months, from about 6 months to about 18 months, from about 6 months to about 15 months, about 15 months, about 18 months, about 21 months, about 24 months, from about 2 years to about 20 years, from about 5 years to about 15 years, from about 5 years to about 10 years, about 3 years, about 4 years, about 5 years, about 10 years, about 15 years, or about 20 years.

The compounds of Formula (I), or pharmaceutically acceptable salts thereof, may be used on their own, but will generally be administered in the form of a pharmaceutical composition in which the Formula (I) compound/salt (active pharmaceutical ingredient (API)) is in a composition comprising a pharmaceutically acceptable adjuvant(s), diluents(s) and/or carrier(s). Conventional procedures for the selection and preparation of suitable pharmaceutical formulations are described in, for example, “Pharmaceuticals—The Science of Dosage Form Designs”, M. E. Aulton, Churchill Livingstone, 2^{nd} Ed. 2002, incorporated by reference herein in its entirety for all purposes.

Depending on the mode of administration, the pharmaceutical composition will comprise from about 0.05 to about 99 wt %, for example, from about 0.05 to about 80 wt %, or from about 0.10 to about 70 wt %, or from about 0.10 to about 50 wt %, of API, all percentages by weight being based on the total weight of the pharmaceutical composition. Unless otherwise provided herein, API weight percentages provided herein are for the respective free base form of the compound of Formula (I).

In one oral administration embodiment, the oral dosage form is a film-coated oral tablet. In a further embodiment, the dosage form is an immediate release dosage form with rapid dissolution characteristics under in vitro test conditions.

In one embodiment, the oral dosage form is administered once daily. In a further embodiment, the oral dosage form is administered at approximately the same time every day, e.g., prior to breakfast. In another embodiment, the composition comprising an effective amount of Formula (I) is administered 2× daily. In yet another embodiment, the composition comprising an effective amount of Formula (I) is administered 1× per week, every other day, every third day, 2× per week, 3× per week, 4× per week, or 5× per week.

For oral administration the compound of the disclosure may be admixed with adjuvant(s), diluent(s) or carrier(s), for example, lactose, saccharose, sorbitol, mannitol; starch, for example, potato starch, corn starch or amylopectin; cellulose derivative; binder, for example, gelatine or polyvinylpyrrolidone; disintegrant, for example cellulose derivative, and/or lubricant, for example, magnesium stearate, calcium stearate, polyethylene glycol, wax, paraffin, and the like, and then compressed into tablets. If coated tablets are required, the cores, prepared as described above, may be coated with a suitable polymer dissolved or dispersed in water or readily volatile organic solvent(s). Alternatively, the tablet may be coated with a concentrated sugar solution which may contain, for example, gum arabic, gelatine, talcum and titanium dioxide.

For the preparation of soft gelatine capsules, the compound of the disclosure may be admixed with, for example, a vegetable oil or polyethylene glycol. Hard gelatine capsules may contain granules of the compound using pharmaceutical excipients like the above-mentioned excipients for tablets. Also, liquid or semisolid formulations of the compound of the disclosure may be filled into hard gelatine capsules.

In one embodiment, the composition is an oral disintegrating tablet (ODT). ODTs differ from traditional tablets in that they are designed to be dissolved on the tongue rather than swallowed whole.

In one embodiment, the composition is an oral thin film or an oral disintegrating film (ODF). Such formulations, when placed on the tongue, hydrate via interaction with saliva, and releases the active compound from the dosage form. The ODF, in one embodiment, contains a film-forming polymer such as hydroxypropylmethylcellulose (HPMC), hydroxypropyl cellulose (HPC), pullulan, carboxymethyl cellulose (CMC), pectin, starch, polyvinyl acetate (PVA) or sodium alginate.

Liquid preparations for oral application may be in the form of syrups, solutions or suspensions. Solutions, for example may contain the compound of the disclosure, the balance being sugar and a mixture of ethanol, water, glycerol and propylene glycol. Optionally such liquid preparations may contain coloring agents, flavoring agents, saccharine and/or carboxymethylcellulose as a thickening agent. Furthermore, other excipients known to those skilled in art may be used when making formulations for oral use.

In one embodiment of the methods, the pharmaceutical composition is one of the compositions described in International Application Publication No. WO 2019/166626, the disclosure of which is incorporated herein by reference in its entirety for all purposes.

In another embodiment of the methods, the pharmaceutical composition administered to the patient is Composition (A) comprising:
(a) from about 1 to about 30 wt % of the compound of Formula (I), or a pharmaceutically acceptable salt thereof;
(b) from about 45 to about 85 wt % of a pharmaceutical diluent;
(c) from about 6 to about 30 wt % of a compression aid;
(d) from about 1 to about 15 wt % of a pharmaceutical disintegrant;
(e) from about 0.00 to about 2 wt % of a pharmaceutical glidant; and
(f) from about 1 to about 10 wt % of a pharmaceutical lubricant;
wherein the components add up to 100 wt %.

In a further embodiment, the compound of Formula (I) is brensocatib. In one embodiment, brensocatib is in polymorphic Form A. In another embodiment, brensocatib is characterized by one of the X-ray powder diffraction patterns described above.

In some embodiments of the methods, Composition (A) comprises the compound of Formula (I), e.g., brensocatib, in an amount from about 1 to about 25 wt %; from about 1 to about 20 wt %; from about 1 to about 15 wt %; from about 1 to about 10 wt %; from about 1 to about 5 wt %, or from about 1 to about 3 wt % of the total weight of the composition.

In some embodiments of the methods, Composition (A) comprises the compound of Formula (I), e.g., brensocatib, in an amount from about 1.5 to about 30 wt %; from about 1.5 to about 25 wt %; from about 1.5 to about 20 wt %; from about 1.5 to about 15 wt %; from about 1.5 to about 10 wt %; or from about 1.5 to about 5 wt % of the total weight of the composition.

In some embodiments of the methods, Composition (A) comprises the compound of Formula (I), e.g., brensocatib, in an amount from about 3 to about 30 wt %; from about 3 to about 25 wt %; from about 3 to about 20 wt %; from about 3 to about 15 wt %; from about 3 to about 10 wt %; or from about 3 to about 5 wt % of the total weight of the composition. In a further embodiment, the compound of Formula (I) is present at from about 3 to about 10 wt % of the total weight of the composition. In a further embodiment, the compound of Formula (I) is brensocatib, or a pharmaceutically acceptable salt thereof.

In some embodiments of the methods, Composition (A) comprises the compound of Formula (I), e.g., brensocatib, in an amount of about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, about 29 wt % or about 30 wt % of the total weight of the composition.

In some embodiments of the methods, Composition (A) comprises the compound of Formula (I), e.g., brensocatib, in an amount of about 5 mg to about 70 mg, or about 10 mg to about 40 mg, for example, 5 mg, 10 mg, 15 mg, 20 mg, 25 mg, 30 mg, 35 mg, 40 mg, 45 mg, 50 mg, or 65 mg. In a further embodiment, Composition (A) comprises the compound of Formula (I) in an amount of 10 mg, 25 mg or 40 mg. In even a further embodiment, the compound of Formula (I) is brensocatib, or a pharmaceutically acceptable salt thereof.

In some embodiments of the methods, Composition (A) comprises one or more pharmaceutical diluents selected from the group consisting of microcrystalline cellulose, calcium carbonate, calcium phosphate, calcium sulfate, cellulose acetate, erythritol, ethylcellulose, fructose, inulin, isomalt, lactitol, lactose, magnesium carbonate, magnesium oxide, maltitol, maltodextrin, maltose, mannitol, polydextrose, polyethylene glycol, pullulan, simethicone, sodium bicarbonate, sodium carbonate, sodium chloride, sorbitol, starch, sucrose, trehalose, xylitol, and a combination of the foregoing. In one embodiment, Composition (A) comprises two or more pharmaceutical diluents. In another embodiment, Composition (A) comprises one pharmaceutical diluent. In a further embodiment, the pharmaceutical diluent is microcrystalline cellulose. Microcrystalline cellulose is a binder/diluent in oral tablet and capsule formulations and can be used in dry-granulation, wet-granulation, and direct-compression processes.

In some embodiments of the methods, Composition (A) comprises one or more pharmaceutical diluents in an amount from about 45 to about 80 wt %, from about 45 to about 75 wt %, from about 45 to about 70 wt %, from about 45 to about 65 wt %, from about 45 to about 60 wt %, or from about 45 to about 55 wt % of the total weight of the composition. In a further embodiment, the one or more pharmaceutical diluents comprises microcrystalline cellulose. In even a further embodiment, the compound of Formula (I) is brensocatib, or a pharmaceutically acceptable salt thereof.

In some embodiments of the methods, Composition (A) comprises one or more pharmaceutical diluents in an amount from about 50 to about 85 wt %, from about 50 to about 75 wt %, from about 55 to about 85 wt %, from about 55 to about 70 wt %, from about 60 to about 85 wt %, from about 65 to about 85 wt %, from about 70 to about 85 wt %, or from about 75 to about 85 wt % of the total weight of the composition. In a further embodiment, the one or more pharmaceutical diluents is present at from about 55 to about 70 wt % of the total weight of the composition. In a further embodiment, the one or more pharmaceutical diluents comprises microcrystalline cellulose. In even a further embodiment, the compound of Formula (I) is brensocatib, or a pharmaceutically acceptable salt thereof.

In some embodiments of the methods, Composition (A) comprises one or more pharmaceutical diluents in an amount of about 45 wt %, about 50 wt %, about 55 wt %, about 60 wt %, about 65 wt %, about 70 wt %, about 75 wt %, about 80 wt % or about 85 wt % of the total weight of the composition.

In some embodiments of the methods, the one or more pharmaceutical diluents in Composition (A) is microcrystalline cellulose. In other embodiments, the one or more pharmaceutical diluents comprises calcium carbonate, calcium phosphate, calcium sulfate, cellulose acetate, erythritol, ethylcellulose, fructose, inulin, isomalt, lactitol, magnesium carbonate, magnesium oxide, maltitol, maltodextrin, maltose, mannitol, polydextrose, polyethylene glycol, pullulan, simethicone, sodium bicarbonate, sodium carbonate, sodium chloride, sorbitol, starch, sucrose, trehalose and xylitol.

In the present disclosure, the terms “disintegrant” and “disintegrants” are intended to be interpreted in the context of pharmaceutical formulation science. Accordingly, a disintegrant in the Composition (A) may be, for example: alginic acid, calcium alginate, carboxymethylcellulose calcium, chitosan, croscarmellose sodium, crospovidone, glycine, guar gum, hydroxypropyl cellulose, low-substituted hydroxypropyl cellulose, magnesium aluminum silicate, methylcellulose, povidone, sodium alginate, sodium carboxymethylcellulose, sodium starch glycolate, starch, or a combination thereof.

In some embodiments of the methods, the one or more disintegrants in Composition (A) is sodium starch glycolate. In one embodiment, the amount of the disintegrants present in Composition (A) is between 2% and 8% of the total weight of the composition. In a further embodiment, the amount of the disintegrants is about 2 wt %, about 2.5 wt %, about 3 wt %, about 3.5 wt %, about 4 wt % or about 4.5 wt % of the total weight of the composition. The physical properties of sodium starch glycolate, and hence its effectiveness as a disintegrant, are affected by the degree of crosslinkage, extent of carboxymethylation, and purity.

In some embodiments of the methods, the one or more pharmaceutical disintegrants in Composition (A) comprises croscarmellose sodium.

In some embodiments of the methods, Composition (A) comprises one or more pharmaceutical disintegrants in an amount from about 2 to about 14 wt %, from about 2 to about 13 wt %, from about 2 to about 12 wt %, from about 2 to about 11 wt %, from about 2 to about 10 wt %, from about 2 to about 9 wt %, from about 2 to about 8 wt %, from about 2 to about 7 wt %, from about 2 to about 6 wt %, from about 2 to about 5 wt %, from about 3.5 to about 4.5 wt % of the total weight of the composition. In a further embodiment, the one or more pharmaceutical disintegrants is present at from about 3.5 to about 4.5 wt % of the total weight of the pharmaceutical composition. In a further embodiment, the one or more pharmaceutical disintegrants is sodium starch glycolate. In a further embodiment, the one or more pharmaceutical diluents comprises microcrystalline cellulose. In even a further embodiment, the compound of Formula (I) is brensocatib, or a pharmaceutically acceptable salt thereof.

In the present disclosure, the terms “glidants” and “gliding agents” are intended to be interpreted in the context of pharmaceutical formulation science. Accordingly, a glidant in Composition (A) may be, for example: silicon dioxide, colloidal silicon dioxide, powdered cellulose, hydrophobic colloidal silica, magnesium oxide, magnesium silicate, magnesium trisilicate, sodium stearate and talc.

Accordingly, in some embodiments of the methods, the one or more pharmaceutical glidants in Composition (A) is selected from silicon dioxide, colloidal silicon dioxide, powdered cellulose, hydrophobic colloidal silica, magnesium oxide, magnesium silicate, magnesium trisilicate, sodium stearate, talc, or a combination of the foregoing. In one embodiment, the glidant is silicon dioxide. Its small particle size and large specific surface area give it desirable flow characteristics that are exploited to improve the flow properties of dry powders in a number of processes such as tableting and capsule filling. Typical silicon dioxide concentrations for use herein range from about 0.05 to about 1.0 wt %. Porous silica gel particles may also be used as a glidant, which may be an advantage for some formulations, with typical concentrations of 0.25-1%.

In some embodiments of the methods, Composition (A) comprises one or more pharmaceutical glidants in an amount from about 0.00 to about 1.75 wt %; from about 0.00 to about 1.50 wt %; from about 0.00 to about 1.25 wt %; from about 0.00 to about 1.00 wt %; from about 0.00 to about 0.75 wt %; from about 0.00 to about 0.50 wt %; from about 0.00 to about 0.25 wt %; from about 0.00 to about 0.20 wt % of the total weight of the composition. In a further embodiment, the one or more pharmaceutical glidants comprises silicon dioxide. In a further embodiment, the one or more pharmaceutical disintegrants is sodium starch glycolate. In a further embodiment, the one or more pharmaceutical diluents comprises microcrystalline cellulose. In even a further embodiment, the compound of Formula (I) in Composition (A) is brensocatib, or a pharmaceutically acceptable salt thereof.

In some embodiments of the methods, Composition (A) comprises one or more pharmaceutical glidants in an amount from about 0.05 to about 2 wt %; from about 0.05 to about 1.75 wt %; from about 0.05 to about 1.50 wt %; from about 0.05 to about 1.25 wt %; from about 0.05 to about 1.00 wt %; from about 0.05 to about 0.75 wt %; from about 0.05 to about 0.50 wt %; from about 0.05 to about 0.25 wt %; or from about 0.05 to about 0.20 wt % of the total weight of the composition. In a further embodiment, the one or more pharmaceutical glidants is present at from about 0.05 to about 0.25 wt % of the total weight of the composition. In a further embodiment, the one or more pharmaceutical glidants comprises silicon dioxide. In a further embodiment, the one or more pharmaceutical disintegrants is sodium starch glycolate. In a further embodiment, the one or more pharmaceutical diluents comprises microcrystalline cellulose. In even a further embodiment, the compound of Formula (I) in Composition (A) is brensocatib, or a pharmaceutically acceptable salt thereof.

In some embodiments of the methods, Composition (A) comprises one or more pharmaceutical glidants in an amount from about 0.05 to about 2 wt %; from about 0.10 to about 2 wt %; from about 0.2 to about 2 wt %; from about 0.3 to about 2 wt %; or from about 0.40 to about 2 wt % of the total weight of the composition. In a further embodiment, the one or more pharmaceutical glidants comprises silicon dioxide. In a further embodiment, the one or more pharmaceutical disintegrants is sodium starch glycolate. In a further embodiment, the one or more pharmaceutical diluents comprises microcrystalline cellulose. In even a further embodiment, the compound of Formula (I) in Composition (A) is brensocatib, or a pharmaceutically acceptable salt thereof.

In the present disclosure, the terms “lubricant” and “lubricants”, as used herein, are intended to be interpreted in the context of pharmaceutical formulation science. Accordingly, a lubricant may be, for example calcium stearate, glyceryl behenate, glyceryl monostearate, glyceryl palmitostearate, a mixture of behenate esters of glycerine (e.g. a mixture of glyceryl bihenehate, tribehenin and glyceryl behenate), leucine, magnesium stearate, myristic acid, palmitic acid, poloxamer, polyethylene glycol, potassium benzoate, sodium benzoate, sodium lauryl sulfate, sodium stearate, sodium stearyl fumarate, stearic acid, talc, tribehenin and zinc stearate.

Accordingly, in some embodiments of the methods, the one or more pharmaceutical lubricants in Composition (A) are selected from the group consisting of calcium stearate, glyceryl behenate, glyceryl monostearate, glyceryl palmitostearate, a mixture of behenate esters of glycerine (e.g., a mixture of glyceryl bihenehate, tribehenin and glyceryl behenate), leucine, magnesium stearate, myristic acid, palmitic acid, poloxamer, polyethylene glycol, potassium benzoate, sodium benzoate, sodium lauryl sulfate, sodium stearate, sodium stearyl fumarate, stearic acid, talc, tribehenin and zinc stearate. In other embodiments, the one or more pharmaceutical lubricants are selected from the group consisting of calcium stearate, glyceryl behenate, glyceryl monostearate, glyceryl palmitostearate, a mixture of behenate esters of glycerine (e.g., a mixture of glyceryl bihenehate, tribehenin and glyceryl behenate), leucine, magnesium stearate, myristic acid, palmitic acid, poloxamer, polyethylene glycol, potassium benzoate, sodium benzoate, sodium lauryl sulfate, sodium stearate, stearic acid, talc, tribehenin and zinc stearate.

In some embodiments of the methods, Composition (A) comprises one or more pharmaceutical lubricants and the lubricant is not sodium stearyl fumarate. In a further embodiment, the compound of Formula (I) in Composition (A) is brensocatib, or a pharmaceutically acceptable salt thereof.

In one embodiment of the methods, Composition (A) includes glycerol behenate as the lubricant.

In some embodiments of the methods, the one or more pharmaceutical lubricants in Composition (A) comprises glyceryl behenate, magnesium stearate, stearic acid, or a combination thereof.

In one embodiment of the methods, the lubricant in Composition (A) is glyceryl behenate, magnesium stearate, or a combination thereof.

In one embodiment of the methods, the one or more pharmaceutical lubricants in Composition (A) comprises sodium stearyl fumarate and/or one or more behenate esters of glycerine.

In some embodiments of the methods, Composition (A) comprises one or more pharmaceutical lubricants in an amount from about 1 wt % to about 9 wt %, from about 1 wt % to about 8 wt %, from about 1 wt % to about 7 wt %, from about 1 wt % to about 6 wt %, from about 1 wt % to about 5 wt %, from about 2 wt % to about 10 wt %, from about 2.5 wt % to about 10 wt %, from about 2 wt % to about 8 wt %, from about 2 wt % to about 7 wt %, from about 2 wt % to about 6 wt %, from about 2 wt % to about 5 wt %, from about 2 wt % to about 4.5 wt %, or from about 2.5 wt % to about 4.5 wt % of the total weight of the composition. In a further embodiment, the one or more pharmaceutical lubricants is present at from about 2.5 to about 4.5 wt % of the total weight of the composition. In a further embodiment, the one or more pharmaceutical lubricants in Composition (A) is glycerol behenate. In a further embodiment, the one or more pharmaceutical glidants in Composition (A) comprises silicon dioxide. In a further embodiment, the one or more pharmaceutical disintegrants in Composition (A) is sodium starch glycolate. In a further embodiment, the one or more pharmaceutical diluents in Composition (A) comprises microcrystalline cellulose. In even a further embodiment, the compound of Formula (I) in Composition (A) is brensocatib, or a pharmaceutically acceptable salt thereof.

In one embodiment of the methods, the one or more pharmaceutical lubricants in Composition (A) consists of sodium stearyl fumarate and/or one or more behenate esters of glycerine or a mixture thereof.

In another embodiment of the methods, the one or more pharmaceutical lubricants in Composition (A) consists of sodium stearyl fumarate, glyceryl dibehenate, glyceryl behenate, tribehenin or any mixture thereof.

In one embodiment of the methods, the one or more pharmaceutical lubricants in Composition (A) comprises sodium stearyl fumarate. In another embodiment, the one or more pharmaceutical lubricants in Composition (A) consists of sodium stearyl fumarate.

In one embodiment of the methods, the one or more pharmaceutical lubricants in Composition (A) comprises one or more behenate esters of glycerine. (i.e., one or more of glyceryl dibehenate, tribehenin and glyceryl behenate).

In one embodiment of the methods, the compression aid in Composition (A) is dicalcium phosphate dihydrate (also known as dibasic calcium phosphate dihydrate) (DCPD). DCPD is used in tablet formulations both as an excipient and as a source of calcium and phosphorus in nutritional supplements.

In one embodiment of the methods, Composition (A) comprises the compression aid, e.g., DCPD, in an amount from about 10 to about 30 wt %, including about 16 wt %, about 17 wt %, about 18 wt %, about 19 wt %, about 20 wt %, about 21 wt %, about 22 wt %, about 23 wt %, or about 24 wt % of the total weight of the composition. In a further embodiment, the compression aid is present at about 20 wt % of the total weight of the composition.

In one embodiment of the methods, Composition (A) comprises the compression aid, e.g., DCPD, in an amount from about 10 to about 25 wt %, from about 10 to about 20 wt %, from about 10 to about 15 wt %, from about 15 to about 25 wt %, or from about 20 to about 25 wt %, or from about 18 to about 22 wt % of the total weight of the composition. In a further embodiment, the compression aid is present at from about 18 to about 22 wt % of the total weight of the composition. In a further embodiment, the compression aid is DCPD. In a further embodiment, the one or more pharmaceutical lubricants in Composition (A) is glycerol behenate. In a further embodiment, the one or more pharmaceutical glidants in Composition (A) comprises silicon dioxide. In a further embodiment, the one or more pharmaceutical disintegrants in Composition (A) is sodium starch glycolate. In a further embodiment, the one or more pharmaceutical diluents in Composition (A) comprises microcrystalline cellulose. In even a further embodiment, the compound of Formula (I) in the exemplary composition is brensocatib, or a pharmaceutically acceptable salt thereof.

In one embodiment of the methods, the pharmaceutical composition administered to the patient is Composition (B) comprising:
(a) from about 1 to about 30 wt % of the compound of Formula (I), or a pharmaceutically acceptable salt thereof;
(b) from about 55 to about 75 wt % of a pharmaceutical diluent;
(c) from about 15 to about 25 wt % of a compression aid;
(d) from about 3 to about 5 wt % of a pharmaceutical disintegrant;
(e) from about 0.00 to about 1 wt % of a pharmaceutical glidant; and
(f) from about 2 to about 6 wt % of a pharmaceutical lubricant;
wherein the components add up to 100 wt %.

In some embodiments of the methods where Composition (B) is administered to the patient, the identity of the pharmaceutical diluent, compression aid, pharmaceutical disintegrant, pharmaceutical glidant, and pharmaceutical lubricant in the composition may be one of those described above for Composition (A). In other embodiments, the amount of the pharmaceutical diluent, compression aid, pharmaceutical disintegrant, pharmaceutical glidant, and pharmaceutical lubricant in Composition (B) may also be one of those described above for Composition (A), as long as the amount is within the corresponding broader range recited above for Composition (B).

The pharmaceutical compositions disclosed herein, including Compositions (A) and (B), may be in a solid dosage form suitable for oral administration to a human being. For example, the pharmaceutical composition is a pharmaceutical tablet. Pharmaceutical tablets may be prepared using methods known to those skilled in the art including, for example, dry mixing/direct compression process as described in International Application Publication No. WO 2019/166626. In some embodiments, the pharmaceutical tablet comprises a tablet core wherein the tablet core comprises the pharmaceutical composition as disclosed herein and wherein the tablet core has a coating. In some embodiments, the coating is a film coating. The film coating may be applied using conventional methods known to those skilled in the art. A functional coating can be used to provide protection against, for example, moisture ingress or degradation by light. Additionally, a functional coating may be used to modify or control the release of the compound of Formula (I), e.g., brensocatib, from the composition. The coating may comprise, for example, about 0.2 to about 10 wt % of the total weight of the pharmaceutical composition, e.g., from about 0.2 to about 4 wt %, from about 0.2 to about 3 wt %, from about 1 to about 6 wt %, or from about 2 to about 5 wt % of the total weight of the pharmaceutical composition

The skilled person will recognise that the compounds of the disclosure may be prepared, in known manner, in a variety of ways.

For example, in one embodiment, compounds of Formula (I) are prepared according to the methods set forth in U.S. Pat. No. 9,522,894, incorporated by reference herein in its entirety for all purposes.

It is noted that one or more DPP1 inhibitors other than the compounds of Formula (I), or pharmaceutically acceptable salts thereof, may also be used in place of, or in combination with, the compounds of Formula (I), or pharmaceutically acceptable salts thereof, according to the disclosed treatment methods. Non-limiting examples of DPP1 inhibitors other than the compounds of Formula (I), or pharmaceutically acceptable salts thereof contemplated for use include those disclosed in Chen et al., Journal of Medicinal Chemistry 64(16):11857-11885 (2021); Banerjee et al., Bioorganic & Medicinal Chemistry Letters 47:128202 (2021); Bondebjerg J et al., Bioorg Med Chem. 13:4408-4424 (2005); Bondejberg J et al., Bioorg Med Chem Lett. 16:3614-3617 (2006); Guarino C et al., Biochem Pharmacol. 131:52-67 (2017); Guay D et al., Bioorg Med Chem Lett. 19:5392-5396 (2009); Guay D et al., Curr Top Med Chem. 10:708-716 (2010); Méthot N et al., J Biol Chem. 282:20836-20846 (2007); Méthot N et al., Mol. Pharm. 73:1857-1865 (2008); Miller et al., Br J Clin Pharmacol. 83:2813-2820 (2017); U.S. Pat. Nos. 8,871,783, 8,877,775, 8,889,708, 8,987,249, 8,999,975, 9,073,869, 9,440,960, 9,713,606, 9,879,026, RE47,636E1, 10,238,633, 9,856,228, and 10,479,781; Chinese Patent Application No: CN202110129457.2A; each of which is incorporated herein by reference in its entirety for all purposes.

In another aspect, the present disclosure provides the diastereomers of brensocatib disclosed herein, i.e., (2R)—N-{(1R)-1-Cyano-2-[4-(3-methyl-2-oxo-2,3-dihydro-1,3-benzoxazol-5-yl)phenyl]ethyl}-1,4-oxazepane-2-carboxamide (i.e., the R,R isomer), (2S)—N-{(1R)-1-Cyano-2-[4-(3-methyl-2-oxo-2,3-dihydro-1,3-benzoxazol-5-yl)phenyl]ethyl}-1,4-oxazepane-2-carboxamide (i.e., the S,R isomer), and (2R)—N-{(1S)-1-Cyano-2-[4-(3-methyl-2-oxo-2,3-dihydro-1,3-benzoxazol-5-yl)phenyl]ethyl}-1,4-oxazepane-2-carboxamide (i.e., the R,S isomer), and their respective pharmaceutically acceptable salts, as well as mixtures comprising brensocatib, or a pharmaceutically acceptable salt thereof, and one or more of the diastereomers of brensocatib or pharmaceutically acceptable salts thereof. In one embodiment, the mixture comprises brensocatib, or a pharmaceutically acceptable salt thereof, and the R,R isomer, or a pharmaceutically acceptable salt thereof. In another embodiment, the mixture comprises brensocatib, or a pharmaceutically acceptable salt thereof, and the S,R isomer, or a pharmaceutically acceptable salt thereof. In still another embodiment, the mixture comprises brensocatib, or a pharmaceutically acceptable salt thereof, and the R,S isomer, or a pharmaceutically acceptable salt thereof.

### EXAMPLES

The present invention is further illustrated by reference to the following Examples. However, it should be noted that the Examples, like the embodiments described above, are illustrative and are not to be construed as restricting the scope of the invention in any way.

### Example 1—Efficacy, Safety and Tolerability, and Pharmacokinetics of Brensocatib Administered Once Daily for 28 Days in Patients with Cystic Fibrosis

Cystic fibrosis is caused by abnormalities in the CF transmembrane conductance regulator protein, causing chronic lung infections (particularly with Pseudomonas aeruginosa) and excessive inflammation, and leading to bronchiectasis, declining lung function, respiratory insufficiency and quality of life. This example describes a Phase 2a randomized single-blind placebo-controlled parallel-group study to assess efficacy, safety, tolerability, and pharmacokinetics of brensocatib tablets in adults with cystic fibrosis (CF). Brensocatib is the International Nonproprietary Name for (2S)—N-{(1S)-1-cyano-2-[4-(3-methyl-2-oxo-2,3-dihydro-1,3-benzoxazol-5-yl)phenyl]ethyl}-1,4-oxazepane-2-carboxamide

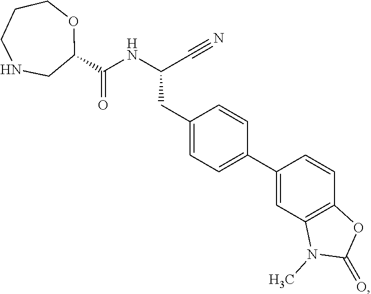 [SMILES: Cn1c(=O)oc2ccc(-c3ccc(C[C@@H](C#N)NC(=O)[C@@H]4CNCCCO4)cc3)cc21]

and will be administered once daily (QD) for 28 days in study participants with CF. Participants are randomized to receive once daily oral dosing of 10 mg brensocatib, 25 mg brensocatib, 40 mg brensocatib, or matching placebo. If appropriate based on the review of the safety and PK data from the above cohorts, an additional cohort will be dosed with 65 mg brensocatib or matching placebo once daily.

Brensocatib film-coated tablets are round, biconvex, brown, film-coated tablets containing the equivalent of 10 mg, 25 mg, and 40 mg of brensocatib drug substance and to be administered orally once daily (QD). Each film-coated tablet contains active ingredient of brensocatib drug substance and compendial ingredients: microcrystalline cellulose, dibasic calcium phosphate dihydrate, sodium starch glycolate, silicon dioxide, and glyceryl behenate. If the 65 mg dose is to be assessed, participants in this cohort will receive 65 mg brensocatib QD (25 mg+40 mg tablets) or matching placebo tablets on the same schedule as the previous cohorts.

Efficacy measures include spirometry, which is a validated method for assessing respiratory function, and the Cystic Fibrosis Questionnaire Revised (CFQ-R) Respiratory Domain, a validated instrument for assessing quality of life for patients with CF. Additionally, administration of brensocatib for 28 days is expected to produce a decrease in NE, PR3, and CatG concentrations in the blood and sputum of participants with CF.

### Subject Eligibility Criteria

Table 1 below provides certain inclusion criteria for the study.

TABLE 1
Inclusion criteria for the study

1. Participants must be ≥18 years of age at the time of signing the
informed consent.
2. Male or female participants with a confirmed diagnosis of CF related
lung disease
a. ppFEV1 between 40% to 90% (inclusive) at Screening Visit and at
Baseline
b. Stable CF treatment for at least 30 days before Screening
3. Participants have a body mass index ≥18 kg/m².
4. Participants are capable of providing informed consent.

Exclusion criteria for the study include severe or unstable CF, as per Investigator's judgement and oxygen saturation (SpO₂) on room air ≤92% at the Screening Visit and at Baseline.

### Study Design

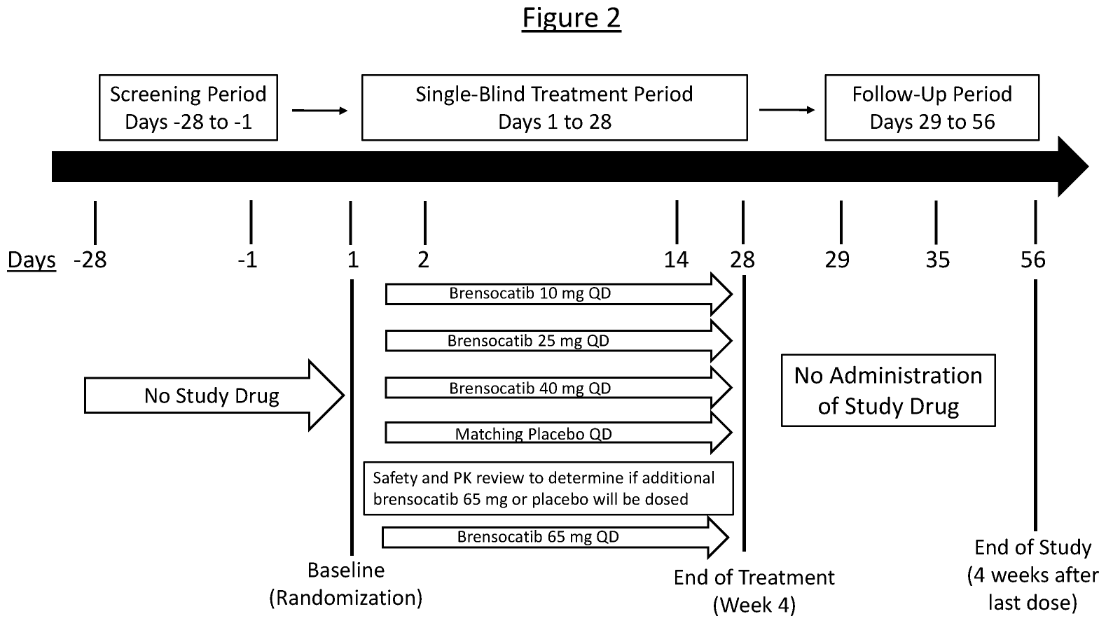

FIG. 2 provides a schematic diagram of the study design and treatment duration. The study will be conducted in a single-blind fashion, i.e., participants are blinded to their assigned study drug and dose, as are the study center staff and the Investigator; select Sponsor personnel, including the Independent Clinical Pharmacologist and Safety Review Committee (SRC) members, are not blinded. Eligible participants with CF will be randomized to receive 10 mg brensocatib, 25 mg brensocatib, 40 mg brensocatib or its matching placebo orally and once daily (QD) for 28 days. Following review of PK and safety data by the SRC, and provided that the SRC deems it appropriate to administer the next dose, a cohort of participants will receive 65 mg brensocatib QD on the same schedule as the previous cohorts. Participants assigned to the 65 mg dose cohort will receive 1 tablet of 25 mg brensocatib and 1 tablet of 40 mg brensocatib.

The study will enroll 36 to 48 participants to form 3-4 cohorts following randomization by means of an Interactive Web Response System (IWRS). Each cohort will enroll 12 participants in a 10:2 ratio (active to placebo). For each cohort, there will be 2 strata: 6 participants (5 active: 1 placebo) who have previously received and will continue to receive cystic fibrosis transmembrane conductance regulator (CFTR) modulators as concomitant medication, and 6 participants (5 active: 1 placebo) who have not and are currently not being treated with CFTR modulators. The first three dose cohorts (a total of 36 participants) will be 10 mg brensocatib, 25 mg brensocatib, and 40 mg brensocatib cohorts. If there are no safety issues in these cohorts, a 65 mg brensocatib dose cohort will be similarly randomized, raising the total sample size from 36 to 48 participants.

This study is designed to investigate the following:
1. The PK effects of 10 mg brensocatib, 25 mg brensocatib, and 40 mg brensocatib QD (and possibly 65 mg brensocatib QD) after 28 days of administration in participants with CF.
2. The PD effects of brensocatib in participants with CF.
3. The safety and tolerability of brensocatib in participants with CF.
   Efficacy will be assessed via spirometry and by CFQ-R in an exploratory manner.

Including the Screening (up to 28 days comprising the first clinic visit (Visit 1)), Treatment (28 days), and Follow-Up Periods (28 days), the study participation duration is expected to be 84 days or less. At the End of Study (EOS) on Day 56, all ongoing AEs will be assessed by the investigators whether they are resolved and stable or not clinically significant. Any study-related safety issues or serious events that extend past Study Day 56 (EOS) will be followed to resolution.

The Treatment Period is a 28-day period comprising 4 in-clinic visits on Day 1 (baseline, Visit 2), Day 2 (Visit 3), Day 14 (Visit 4), and Day 28 (End of Treatment (EOT), Visit 5). Participants must enter the study at the start of either the on-treatment cycle or off-treatment cycle of their inhaled antibiotic regimen. The study center staff will administer the study drug to participants for days of in-clinic visits. On Days 1 and 28, the study center staff will administer the study drug to the participant after 8 hours of fasting on Days 1 and 28. Participants will receive a 28-day supply of blinded study drug on Days 1 and 14. On days when there is no in-clinic visit, the participant will self-administer the study drug with or without food at the same time of day each day.

On Day 1 (baseline), participants will return to the study center after an 8-hour fast and undergo assessments (e.g., inclusion/exclusion criteria, concomitant medications, AEs), and procedures (e.g., physical examination, 12-lead ECG, vital signs, clinical laboratory tests, spirometry). After assessments and procedures are completed, eligible participants will be randomized to receive 10 mg brensocatib, 25 mg brensocatib, or 40 mg brensocatib, or placebo QD, orally for 4 weeks. The first dose of study drug will be administered to the participant by the study center staff on Day 1. Participants will receive a 28-day supply of blinded study drug on Day 1, and will be expected to take the study drug at home, at the same time of day each day, except for days of in-clinic visits, during which study drug will be administered by the study center staff. Participants will return to the study center on Days 2, 14, and 28 (EOT), and report all concomitant medications taken and any AEs that have occurred since the last study visit.

The Follow-Up Period will extend from Day 29 to Day 56 (EOS) and include visits to the study center on Day 29 (Visit 6) and Day 35 (Visit 7) for blood sampling for PK analysis, and for blood and sputum sampling for PD analysis. On Day 56 (EOS) a telephone call will be placed from the study center to the participant to check on well-being and to assess the status of any new and/or ongoing AEs.

The SRC will review safety and PK data of completed cohorts exposed to 10 mg brensocatib, 25 mg brensocatib, and 40 mg brensocatib QD in an unblinded manner to determine whether brensocatib has an acceptable safety and PK profile, and can be escalated to the 65 mg dose. The planned cohort receiving 65 mg brensocatib orally and once daily (QD) will have the same dosing schedule (i.e., QD×28 days) and same allocation rules used in the first 3 cohorts receiving 10 mg brensocatib, 25 mg brensocatib, and 40 mg brensocatib QD, respectively.

### Outcomes Assessment

The objectives and endpoints of the study are shown in Table 2.

TABLE 2
Objectives and endpoints

| Objectives | Endpoints |
|---|---|
| To evaluate the PK of brensocatib in participants | Cₘₐₓ, tₘₐₓ, AUC₀₋₂₄, and t_{1/2} on Day 1 and Day 28 |
| with CF following once daily oral administration of | |
| study drug | |
| To evaluate the safety of brensocatib compared to | Frequency of treatment-emergent adverse |
| placebo in participants with CF over the 4-week | events^{a} |
| treatment period | |
| To evaluate the dose-dependency of brensocatib | Cₘₐₓ, AUC₀₋₂₄, and AUCₗₐₛₜ on Day 1 and Day |
| exposure | 28 |
| To evaluate the effect of brensocatib compared | Change from Baseline to Day 14, Day 28, and |
| with placebo on the concentration of NE, CatG, and | over the 28 day treatment period for concentration |
| PR3 in sputum over the 28 day treatment period | of NE, CatG, and PR3 in sputum |
| To evaluate the effect of brensocatib compared | Change from Baseline to Day 14, Day 28, and |
| with placebo on the concentration of NE, CatG, and | over the 28 day treatment period for concentration |
| PR3 in blood over the 28 day treatment period | of NE, CatG, and PR3 in blood |
| To assess the effect of brensocatib compared with | Change from Baseline to Day 28 in ppFEV₁, |
| placebo on lung function, as measured by change in | FEV₁, FVC, and FEF_{(25-75%)} |
| pulmonary function parameters, including ppFEV₁, | |
| FEV₁, FVC, and FEF_{(25-75%)} at Day 28 | |
| To evaluate the effect of brensocatib compared | Change from Baseline to Day 14 and Day 28 |
| with placebo on quality of life as measured by CFQ- | for the CFQ-R Respiratory Domain score |
| R through Week 4 | |
| To evaluate the PK/PD relationship between | NE, CatG, and PR3 concentrations between |
| AUC/dose and biomarker activity (blood and/or | Day 14 and Day 28 in blood and sputum vs. |
| sputum) | brensocatib exposure at steady state |
| To evaluate the PK/PD relationship between | AESIs during the 28-day treatment period and |
| systemic exposure and safety (e.g., AESIs of | their relationship to brensocatib exposure at steady |
| hyperkeratosis, periodontitis/gingivitis, and infections) | state |
| To evaluate the PK/PD relationship between | ppFEV₁ and CFQ-R during the 28-day |
| systemic exposure and clinical efficacy (i.e., | treatment period and their relationships with |
| ppFEV₁ and CFQ-R) | brensocatib exposure at steady state |
| To evaluate the effect of brensocatib on sputum | Change from Baseline to Day 28 in number of |
| microbiology | colony forming units for microbial species. |

^{a}Frequency of TEAEs includes clinically significant vital signs and laboratory abnormalities
AESI = adverse events of special interest, AUC = area under the concentration-time curve, AUCₗₐₛₜ = AUC from time 0 to the last timepoint with measurable concentration, AUC₀₋₂₄ = AUC from time 0 to 24 h postdose, CatG = cathepsin G, CFQ-R = Cystic Fibrosis Questionnaire-Revised, Cₘₐₓ = maximum plasma concentration, FEF_{(25-75%)} = forced expiratory flow between 25% and 75% of forced vital capacity, FEV₁ = forced expiratory volume in 1 second, FVC = forced vital capacity, NE = neutrophil elastase, PD = pharmacodynamic, PK = pharmacokinetic(s), ppFEV₁ = percent predicted forced expiratory volume in 1 second, PR3 = proteinase 3, t_{1/2} = elimination half-life, tₘₐₓ = time to maximum plasma concentration.

### Pharmacokinetic Assessments

### 1. PK Sample Collection

Blood samples will be collected on Day 1 (Visit 2), Day 2 (Visit 3), 14 (Visit 4), and Day 28 (Visit 5) during the treatment period, and on Day 29 (Visit 6) and Day 35 (Visit 7) during the follow-up period, for PK analysis as well as for measurement of plasma concentrations of brensocatib. Specific timepoints are: Day 1 (predose and at 0.5, 1, 2, 4, 6, 8, and 24 hours postdose (24-hour postdose collection is Day 2 predose collection), Day 14 (predose and at 2 hour postdose), Day 28 (predose and at 0.5, 1, 2, 4, 6, 8, 24 (collected on Day 29), and 168 hours postdose (±24 hours; collected on Day 35). The collection windows will be up to 30 minutes prior to dosing for the predose sample, ±5 minutes for the 0.5, 1, and 2-hour samples, ±15 minutes for the 4-, 6-, and 8-hour samples, ±1 hour for the 24-hour sample, and ±24 hours for the 168-hour sample. Approximately 6 mL of blood is collected at each timepoint.

A maximum of 2 samples may be collected at additional time points during the study if an SAE occurs or at early termination.

### 2. PK Analysis

Individual PK parameters of brensocatib will be determined using noncompartmental analysis for the following parameters: Cₘₐₓ, tₘₐₓ, Cₘᵢₙ, AUC₀₋₂₄, AUCₗₐₛₜ, AUC_{0-∞}, CL/F, Vd/F, t_{1/2}, R_{ac(Cmax)}, and R_{ac(AUC)} on Day 1 and Day 28, as appropriate. C_{trough} on Day 2, Day 14, Day 28, and Day 29 will be evaluated by statistical descriptive values.

Among the PK parameters, the primary endpoints for PK evaluation will be Cₘₐₓ, Tₘₐₓ, AUC₀₋₂₄ and t_{1/2} on Days 1 and 28. A secondary analysis of dose-dependency will be performed based on AUCₗₐₛₜ, AUC₀₋₂₄ and Cₘₐₓ for Day 1 and Day 28. Additional PK parameters, such as AUCₗₐₛₜ, AUC_{inf}, CL/F, Vd/F, Cₘᵢₙ, C_{trough}, R_{ac(AUC)} and R_{ac(Cmax)}, will be determined when data permit. All PK parameters are described in Table 3.

TABLE 3
Pharmacokinetic Endpoints

| Parameter | Definition |
|---|---|
| AUC_{0-∞} | Area under the concentration-time curve from time 0 to |
| | infinity |
| AUC₀₋₂₄ | Area under the concentration-time curve from time 0 to 24 |
| | hours postdose |
| AUCₗₐₛₜ | Area under the concentration-time curve from time 0 to the |
| | last timepoint with measurable concentration |
| CL/F | Apparent total clearance of drug from plasma after |
| | extravascular administration |
| Cₘₐₓ | Maximum plasma concentration |
| Cₘᵢₙ | Minimum plasma concentration |
| C_{trough} | Plasma concentration before the next dose |
| R_{ac(AUC)} | Accumulation ratio based on AUC at steady state (AUC₀₋₂₄ |
| | ratio between Day 28 and Day 1) |
| R_{ac(Cmax)} | Accumulation ratio based on Cₘₐₓ at steady state (Cₘₐₓ ratio |
| | between Day 28 and Day 1) |
| t_{1/2} | Elimination half-life |
| tₘₐₓ | Time to maximum plasma concentration |
| Vd/F | Apparent volume of distribution |

Relationships between PK (brensocatib dose and exposure) and PD effects (e.g., NE concentrations in blood and sputum, ppFEV₁) and safety (e.g., AESI, including hyperkeratosis, periodontitis/gingivitis, and infections) will be explored.

### Biomarkers

6 mL of blood and 3 mL of sputum will be collected from each participant at predose during the screening (Visit 1), on Day 1 (Visit 2), Day 2 (Visit 3), Day 14 (Visit 4), and Day 28 (Visit 5) during the treatment period, and on Day 29 (Visit 6) and Day 35 (Visit 7) during the follow-up period, for biomarker research. Sputum samples will be obtained from participants either spontaneously, with chest physiotherapy, or by induction. Samples will be tested for the effect of brensocatib compared with placebo on the concentration of NE, CatG, and PR3 over the 4-week treatment period and 1 week after the treatment, to evaluate the relationship between study drug dose, AUC, and biomarker activity.

### Microbiology Assessment of Sputum

Any remaining sputum samples from the biomarker study collected on Day 1 and Day 28 will be used for microbiology assessment. Microbial species and colony forming units from sputum assessments will be listed and summarized by brensocatib dose levels and placebo.

### Efficacy Assessments

### 1. Spirometry

Spirometry assessments will be performed during the screening period (Visit 1), and on Day 1 (Visit 2) and Day 28 (Visit 5) during the treatment period, and include the following: prebronchodilator FEV₁, prebronchodilator ppFEV₁, FVC, FEF_{(25-75%)}, and PEFR.

Prebronchodilator pulmonary function tests (PFT) by spirometry (FEV₁, ppFEV₁, FVC, PEFR, and FEF_{(25-75%)}) will be performed per the ATS/ERS criteria (see Miller et al., Eur Respir J. 26(2):319-38 (2005), incorporated herein by reference in its entirety for all purposes). Participants will be provided with detailed instruction on how to conduct the FVC maneuver per ATS/ERS spirometry standardization before performing the test. Time of the last bronchodilator medication use before the procedure will be recorded.

Spirometry will be performed preferably in the morning (AM) at approximately the same time each visit. The same spirometer and standard spirometric techniques, including calibration, will be used to perform spirometry at all visits and, whenever possible, the same person will perform the measurements. Pulmonary function tests will be performed with the participant in a sitting position; however, if necessary to undertake the testing with the participant standing or in another position, this will be noted on the spirometry report. For any participant, the position will be consistent throughout the study. Three measurements fulfilling the ATS acceptability and repeatability criteria will be obtained at every visit. The acceptability criteria will be applied before the repeatability criteria. Unacceptable maneuvers will be discarded before applying the repeatability criteria. If a participant fails to provide repeatable maneuvers, an explanation will be recorded in the source documentation. At least 2 acceptable curves will be obtained.

The largest FEV₁ and largest FVC will be recorded after the data are examined from all of the acceptable curves, even if they do not come from the same curve. The FEF_{(25-75%)} will be obtained from the single curve that meets the acceptability criteria and gives the largest sum of FVC plus FEV₁ (best test). Automated best efforts, which combine FEV₁ and FVC are not acceptable. The spirometer will be calibrated following the principles of the ATS/ERS guidelines every day that a study participant is assessed and spirometry is carried out. The calibration records will be kept in a reviewable log. It is preferred that the calibration equipment (i.e., 3-L syringe) that is used to calibrate the spirometer be subjected to a validated calibration according to the manufacturer's specifications.

Participants will be advised to rest at least 30 minutes and not to eat a large meal for at least 2 hours prior to the test. If a participant is scheduled to have pulmonary rehabilitation on the day of their visit, they will be advised to have the PFT done before the rehabilitation on that day.

### 2. Cystic Fibrosis Questionnaire—Revised (CFQ-R)

Efficacy assessment measured by CFQ-R will be performed on Day 1 (Visit 2), Day 14 (Visit 4), Day 28 (Visit 5) during the treatment period, and on Day 35 (Visit 7) during the follow-up period. CFQ-R will be completed as the first procedure of the visit per FDA guidelines.

Plasma concentrations of brensocatib will be listed and summarized by active dose group and by active dose group within strata over each scheduled sampling time, using descriptive statistics (including arithmetic mean, SD, median, minimum and maximum, geometric mean (GM) with 95% CI, and CV (%) of the GM, as appropriate). Individual plasma concentration data versus time will be presented in data listings, along with graphical plots of individual and GM plasma concentration-time plots presented in linear and semi-logarithmic scales.

The main PK endpoints (Cₘₐₓ, tₘₐₓ, AUC₀₋₂₄, and t_{1/2} on Day 1 and Day 28) will be determined using non-compartmental analysis.

All plasma PK parameters, including the ones that are not included in the primary endpoint, will be listed and summarized using descriptive statistics. All concentrations will be listed and summarized using descriptive statistics based on nominal timepoints.

Analysis of dose dependency for brensocatib AUC₀₋₂₄, AUCₗₐₛₜ, and Cₘₐₓ after single- and multiple-dose administration will be performed using a power law model. The log-transformed PK parameters will be regressed onto log-transformed dose. An estimate of the slope and corresponding 95% CI will be reported. In addition, pairwise comparisons of dose dependency among brensocatib dose levels may be performed based on dose-normalized AUC₀₋₂₄, AUCₗₐₛₜ, and Cₘₐₓ for Day 1 and Day 28.

NE, CatG, and PR3 (in sputum and in blood) concentrations will be measured. The results of these parameters will be summarized by treatment group (dose levels of brensocatib and pooled placebo) and by treatment group within strata based on CFTR modulator use or not. Where sufficient sample sizes are achieved (i.e., adequate sample obtained and minimum number of quantifiable concentrations), a linear model with appropriate covariance pattern will be fit to the exploratory endpoints and between-group comparisons with associated 95% CIs will be reported.

Individual figures of NE, CatG, and PR3 versus time will be presented with all participants overlaid on the same plot for each dose level (spaghetti plots). Mean plots versus time will also be presented by treatment group (dose levels of brensocatib and pooled placebo) and by treatment group within each stratum based on CFTR modulator use or not.

FEV₁ and CFQ-R Respiratory Domain will also be assessed. These variables will be listed and summarized in tables by brensocatib dose levels and placebo.

Additional PK-PD evaluations will be performed in separate analyses, in which the relationship between brensocatib exposure (dose or AUC) and clinical measurements (PD biomarkers, ppFEV₁ and AESI) will be explored.

It is expected that brensocatib treatment will benefit the CF patients by effectively improving the lung function as measured by ppFEV₁, FEV₁, FVC, FEF_{(25-75%)} and PEFR, as well as quality of life as measured by CFQ-R, and reducing the number of colony forming units for microbial species present in the sputum.

### Example 2—Determination of the Therapeutic Effect of Brensocatib on CF Using an In Vitro Transmigration Model and Analysis of Companion Biomarkers

Chronic inflammation driven by abundant dysfunctional polymorphonuclear neutrophils (PMNs) is one of the main causes of the morbidity and mortality in CF. PMNs in the CF airway milieu constitute distinct pathological phenotypes, called GRIM (enhanced granule release leading to neutrophil elastase (NE) exocytosis, immunoregulatory function, and metabolic activities). See Forrest O A et al., J Leukoc Biol. 104(4):665-675 (2018), incorporated herein by reference in its entirety for all purposes. An in vitro transmigration model that recapitulates GRIM fate of PMNs has been developed to understand neutrophil plasticity and functional adaptation. See U.S. Patent Application Publication No. US2020/0256866, incorporated herein by reference in its entirety for all purposes.

The in vitro transmigration model includes a collagen-coated porous 3D scaffold with the H441 club-like small airway cells grown at an air-liquid interface. Blood PMNs that are loaded into a porous scaffold transmigrate through the H441 cells into airway milieu, airway supernatant (ASN), collected from CF patients. Transmigration of PMNs to CF patient ASN showed pathological GRIM phenotypes and functions and expressed similar phenotypes to those found in airway collected from CF patients. The ASN and transmigration are both required for inducing pathological conditioning of PMNs. The ASN of healthy control and individual chemoattractant alone (LTB4, CXCL8, and bacterial products like fMLF and LPS) or direct incubation of blood PMNs in CF ASN without transmigration failed to induce pathological conditioning of PMNs.

In this example, the in vitro transmigration model is used to evaluate the effect of brensocatib treatment on neutrophil precursors and/or in a stem cell-derived neutrophil model. Brensocatib is a potent inhibitor of dipeptidyl peptidase 1 (DPP1) that activates neutrophil serine proteases (NSPs), such as NE, in the promyelocyte stage of neutrophil development in the bone marrow. In the study, PMNs are differentiated from neutrophil precursors and/or stem cell-derived neutrophil model in the presence or absence of brensocatib. The differentiated PMNs are then tested using the in vitro transmigration model to measure their NSP levels, phenotypes and functions before and after transmigrating into the airway milieu (e.g. ASN) of CF patients.

The HL-60 cell line is a model of neutrophil precursors. HL-60 cells are at the myeloblast stage of development and are induced to differentiate terminally to a neutrophil-like state with differentiating inducers. These inducers can be combined with or without brensocatib, followed by applying the cell culture to the CF in vitro transmigration model to evaluate the effect of brensocatib on CF.

Stem cell-derived neutrophil models are also used for the study. Hematopoietic stem cells are differentiated to myeloblast and further to neutrophils with cytokines. To evaluate the pharmacological effects of brensocatib, stem cells will be differentiated to neutrophils in the absence or presence of brensocatib. Primary bone marrow- or umbilical cord blood-derived CD34+ neutrophil progenitor cells are cultured for 7 days in specific stem cell media supplemented with recombinant human Stem Cell Factor and recombinant human IL-3. The cells will be differentiated in culture for another 7 days in the stem cell media with recombinant human Granulocyte Colony Stimulating Factor, plus increasing concentrations of brensocatib. At the end of the differentiation/treatment period, those cells will be applied to the CF in vitro transmigration model.

In vitro transmigration model will also be used to test rodent PMNs. The rodent PMNs will be obtained from wild type rodents treated with brensocatib, or from a DPP1 deficient (DPPT−/−) rodent model.

Lastly, human PMNs obtained from either CF or non-CF patients, or PMNs obtained from animal models of human genetic disorders treated with brensocatib will also be tested using the CF in vitro transmigration model.

Various biomarker and functional analyses will be performed in the above-mentioned in vitro model studies, including (1) granule release to evaluate whether there is increased NE release via primary granule exocytosis (CD63), or a decrease in the surface phagocytic receptor (CD16); (2) immunoregulatory function for modulating T-cell inhibitory molecules, such as increased Arg1 and bimodal PD-L1 expression; (3) metabolic activities such as increased surface Glut1 expression, glycolysis (extracellular acidification rate, ECAR), oxygen consumption (oxygen consumption rate, OCR), ROS production (CellRox), and extracellular lactate levels; and (4) a decrease in bacterial killing. It will be evaluated whether brensocatib has any effect on altering these biomarker levels or relevant functions.

### Example 3—Determination of the Therapeutic Effect of Brensocatib on CF and Associated Neutrophil Extracellular Trap (NET) Formation Using the β Subunit of the Epithelial Sodium Channel (βENaC)-Overexpressing Transgenic Mice and Analysis of Companion Biomarkers

Chronic inflammation driven by abundant dysfunctional polymorphonuclear neutrophils (PMNs), an increase in active neutrophil serine protease (NSP) levels, and the increased formation of neutrophil extracellular traps (NETs) are associated with increased morbidity and mortality in CF.

Both DNA and NETs have been detected in the lungs of CF patients. Free DNA in CF airways has been correlated with reduced lung function, as well as increased levels of neutrophil-recruiting chemokines, and risk of infection. See Manzenreiter R et al., “Ultrastructural characterization of cystic fibrosis sputum using atomic force and scanning electron microscopy,” J Cyst Fibros. 11:84-92 (2012); Marcos V et al., “Free DNA in cystic fibrosis airway fluids correlates with airflow obstruction,” Mediat Inflamm. 2015:1-11 (2015); Dwyer M et al., “Cystic fibrosis sputum DNA has NETosis characteristics and neutrophil extracellular trap release is regulated by macrophage migration inhibitory factor,” J Innate Immun. 6:765-79 (2014), each of which is incorporated herein by reference in their entirety for all purposes. Both laboratory isolates and CF clinical isolates of Pseudomonas aeruginosa, a key bacterial agent of CF lung infections and one of the most important pathogens in progressive and severe CF lung disease, strongly trigger NET release. See Yoo D, et al., “Release of cystic fibrosis airway inflammatory markers from Pseudomonas aeruginosa—stimulated human neutrophils involves NADPH oxidase-dependent extracellular DNA trap formation,” J Immunol. 192:4728-38 (2014); Yoo D G et al., “NET formation induced by Pseudomonas aeruginosa cystic fibrosis isolates measured as release of myeloperoxidase-DNA and neutrophil elastase-DNA complexes,” Immunol Lett. 160:186-94 (2014); Floyd M et al., “Swimming motility mediates the formation of neutrophil extracellular traps induced by flagellated Pseudomonas aeruginosa,” PLoS Pathog. 12:e1005987 (2016), each of which is incorporated herein by reference in their entirety for all purposes. Adult CF patients develop an autoimmune response against NET components that correlates with worsening of lung disease. See Yadav R et al., “Systemic levels of anti-PAD4 autoantibodies correlate with airway obstruction in cystic fibrosis,” J Cyst Fibros. 18:636-45 (2019), incorporated herein by reference in its entirety for all purposes.

Additionally, NETs are present at higher levels in the airways of the β subunit of the epithelial sodium channel (βENaC)-overexpressing transgenic (PENaC-Tg) mice with CF-like lung disease than in wild type mice. See Tucker S L et al., “Neutrophil extracellular traps are present in the airways of ENaC-overexpressing mice with cystic fibrosis-like lung disease,” BMC Immunol. 22(1):7 (2021), incorporated herein by reference in its entirety for all purposes. Tucker S L et al. have assessed NET formation by the presence of myeloperoxidase (MPO)-DNA complexes in bronchioalveolar lavage fluid (BAL) of adult PENaC-Tg and wild type (WT) mice at 6 and 8 weeks of age, and confirmed NET formation by immunofluorescence imaging of BAL cells isolated from both mouse strains. Co-localization of MPO, citrullinated histone 3 (CitH3), a hallmark of PAD4-dependent NET release, and DNA (DAPI) was observed that is indicative of NET release in the PENaC-Tg mice. In contrast, only minimal MPO and CitH3 staining was detected in BAL cells from WT mice using the same microscope settings. Flow cytometry was used to quantify the presence of neutrophils undergoing PAD4-mediated NET release. There were significantly more CitH3 positive cells/ml in the BAL of PENaC-Tg mice at both ages compared to the WT controls. Studies of the PENaC-Tg mice also demonstrated that increased airway sodium absorption causes airway surface liquid depletion, reduced mucus transport, and spontaneous CF-like lung disease with airway mucus obstruction, impaired mucociliary clearance, emphysema, and chronic inflammation including airway neutrophilia, similar to human CF lung disease.

Brensocatib is a potent inhibitor of DPP1 that activates NSPs, which are linked to NET formation. In this example, βENaC-Tg mice with CF-like lung disease are used to evaluate the effect of brensocatib treatment on NET formation in the lungs. In the study, βENaC-Tg mice are exposed to brensocatib or placebo QD or BID for at least one week prior to the evaluation of NETs using, for example, the methods described in Tucker S L et al., mentioned above. WT mice are used as a control for baseline NET formation in the airways. Reduction in the formation of NETs in βENaC-Tg mice by brensocatib vs. the placebo may indicate a significant role of brensocatib in attenuating CF.

Additionally, various biomarker and functional analyses will be performed in the above-mentioned study using the βENaC-Tg mice, including (1) histopathology evaluation of lungs at 6 and 8 weeks of age to assess immune cell recruitment and presence and severity of lesions; (2) immunohistochemistry evaluation to allow for imaging of presence of NSPs and MPO; (3) flow cytometry to characterize myeloid cell subsets in BAL as well as the amount of histone citrullination in the airways from BAL samples; (4) evaluation of MPO, NSPs, MPO-DNA complex, NE-DNA complex, and other NSP-DNA complex biomarkers in the serum by ELISA; (5) soluble protein analytes of BAL samples for cytokine analysis; and (6) immunofluorescence assay of the BAL samples to detect MPO or histone H3. It will be evaluated whether brensocatib has any effect on altering these biomarker levels or relevant functions, or how long the animals need to be treated with brensocatib to show a significant effect.

All, documents, patents, patent applications, publications, product descriptions, and protocols which are cited throughout this application are incorporated herein by reference in their entireties for all purposes.

The embodiments illustrated and discussed in this specification are intended only to teach those skilled in the art the best way known to the inventors to make and use the invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

1. A method for treating cystic fibrosis (CF) in a patient in need of treatment, comprising, administering to the patient for an administration period, a pharmaceutical composition comprising an effective amount of a compound of Formula (I), or a pharmaceutically acceptable salt thereof, 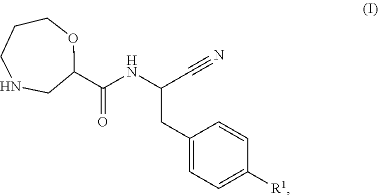
wherein,
R¹ is 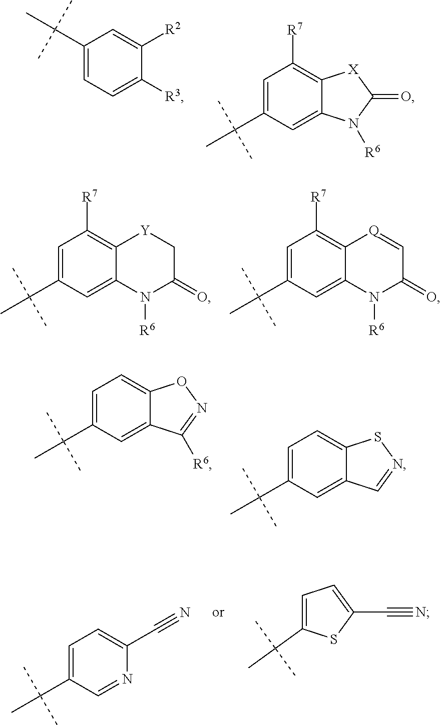
R² is hydrogen, F, Cl, Br, OSO₂C₁₋₃alkyl, or C₁₋₃alkyl;
R³ is hydrogen, F, Cl, Br, CN, CF₃, SO₂C₁₋₃alkyl, CONH₂ or SO₂NR⁴R⁵,
wherein R⁴ and R⁵ together with the nitrogen atom to which they are attached form an azetidine, pyrrolidine or piperidine ring;
X is O, S or CF₂;
Y is O or S;
Q is CH or N;
R⁶ is C₁₋₃alkyl, wherein the C₁₋₃alkyl is optionally substituted by 1, 2 or 3 F and optionally by one substituent selected from OH, OC₁₋₃alkyl, N(C₁₋₃alkyl)₂, cyclopropyl, or tetrahydropyran; and
R⁷ is hydrogen, F, Cl or CH₃;
wherein treating comprises (i) improving the lung function of the patient, as compared to the lung function of the patient prior to the administration period; (ii) improving the patient's quality of life (QOL) assessed by the cystic fibrosis questionnaire-revised (CFQ-R), as compared to the patient's QOL assessed by the CFQ-R prior to the administration period; or (iii) both (i) and (ii).

2. The method of claim 1, wherein the compound of Formula (I), or a pharmaceutically acceptable salt thereof, is the S,S diastereomer:

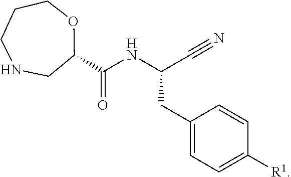

3. The method of claim 1, wherein the compound of Formula (I), or a pharmaceutically acceptable salt thereof, is the S,R diastereomer:

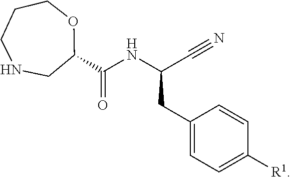

4. The method of claim 1, wherein the compound of Formula (I), or a pharmaceutically acceptable salt thereof, is the R,S diastereomer:

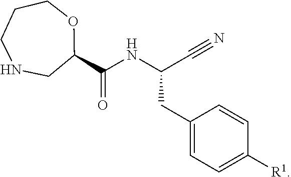

5. The method of claim 1, wherein the compound of Formula (I), or a pharmaceutically acceptable salt thereof, is the R,R diastereomer:

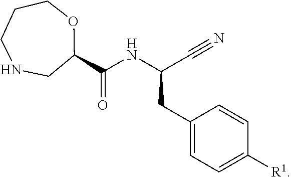

6. The method of claim 1, wherein the composition comprises a mixture of an S,S diastereomer of a compound of Formula (I) and an S,R diastereomer of a compound of Formula (I).

7. The method of claim 1, wherein the composition comprises a mixture of an S,S diastereomer of a compound of Formula (I) and an R,S diastereomer of a compound of Formula (I).

8. The method of claim 1, wherein the composition comprises a mixture of an S,S diastereomer of a compound of Formula (I) and an R,R diastereomer of a compound of Formula (I).

9. The method of any one of claims 1-8, wherein R¹ is 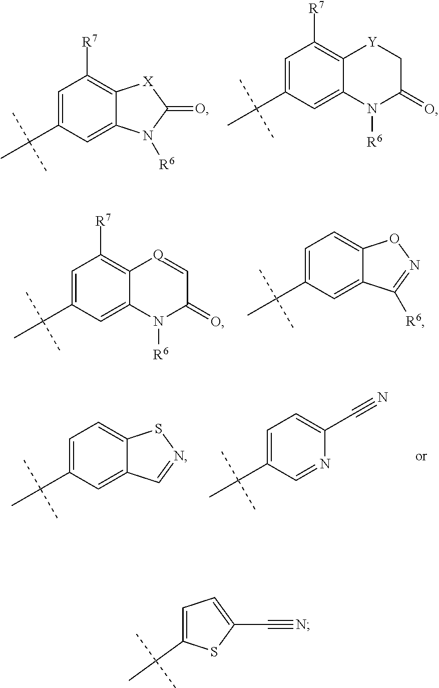
X is O, S or CF₂;
Y is O or S;
Q is CH or N;
R⁶ is C₁₋₃alkyl, wherein the C₁₋₃alkyl is optionally substituted by 1, 2 or 3 F and optionally by one substituent selected from OH, OC₁₋₃alkyl, N(C₁₋₃alkyl)₂, cyclopropyl, or tetrahydropyran; and
R⁷ is hydrogen, F, Cl or CH₃.

10. The method of any one of claims 1-9, wherein,
R¹ is 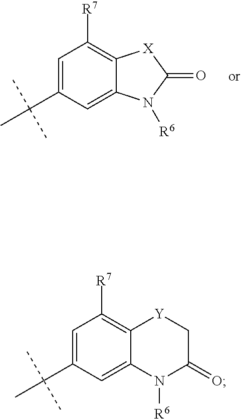
X is O, S or CF₂;
Y is O or S;
R⁶ is C₁₋₃alkyl, wherein the C₁₋₃alkyl is optionally substituted by 1, 2 or 3 F and optionally by one substituent selected from OH, OC₁₋₃alkyl, N(C₁₋₃alkyl)₂, cyclopropyl, or tetrahydropyran; and
R⁷ is hydrogen, F, Cl or CH₃.

11. The method of any one of claims 1-10, wherein, R¹ is

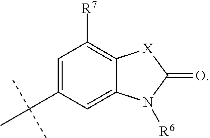

12. The method of any one of claims 1-11, wherein X is O, S or CF₂; R⁶ is C₁₋₃alkyl, wherein the C₁₋₃alkyl is optionally substituted by 1, 2 or 3 F; and R⁷ is hydrogen, F, Cl or CH₃.

13. The method of any one of claims 1-11, wherein X is O; R⁶ is C₁₋₃alkyl, wherein the C₁₋₃alkyl is optionally substituted by 1, 2 or 3 F; and R⁷ is hydrogen.

14. The method of any one of claims 1-11, wherein X is O; R⁶ is C₁₋₃alkyl; and R⁷ is hydrogen.

15. The method of claim 1 or 2, wherein the compound of Formula (I) is selected from the group consisting of
(2S)—N-[(1S)-1-Cyano-2-(4′-cyanobiphenyl-4-yl)ethyl]-1,4-oxazepane-2-carboxamide;
(2S)—N-{(1S)-1-Cyano-2-[4-(3-methyl-2-oxo-2,3-dihydro-1,3-benzoxazol-5-yl)phenyl]ethyl}-1,4-oxazepane-2-carboxamide;
(2S)—N-{(1S)-1-Cyano-2-[4-(3,7-dimethyl-2-oxo-2,3-dihydro-1,3-benzoxazol-5-yl)phenyl]ethyl}-1,4-oxazepane-2-carboxamide;
4′-[(2S)-2-Cyano-2-{[(2S)-1,4-oxazepan-2-ylcarbonyl]amino}ethyl]biphenyl-3-yl methanesulfonate;
(2S)—N-{(1S)-1-Cyano-2-[4-(3-methyl-1,2-benzoxazol-5-yl)phenyl]ethyl}-1,4-oxazepane-2-carboxamide;
(2S)—N-{(1S)-1-Cyano-2-[4′-(trifluoromethyl)biphenyl-4-yl]ethyl}-1,4-oxazepane-2-carboxamide;
(2S)—N-[(1S)-1-Cyano-2-(3′,4′-difluorobiphenyl-4-yl)ethyl]-1,4-oxazepane-2-carboxamide;
(2S)—N-{(1S)-1-Cyano-2-[4-(6-cyanopyridin-3-yl)phenyl]ethyl}-1,4-oxazepane-2-carboxamide;
(2S)—N-{(1S)-1-Cyano-2-[4-(4-methyl-3-oxo-3,4-dihydro-2H-1,4-benzothiazin-6-yl)phenyl]ethyl}-1,4-oxazepane-2-carboxamide;
(2S)—N-{(1S)-1-Cyano-2-[4-(3-ethyl-7-methyl-2-oxo-2,3-dihydro-1,3-benzoxazol-5-yl)phenyl]ethyl}-1,4-oxazepane-2-carboxamide;
(2S)—N-[(1S)-1-Cyano-2-{4-[3-(2-hydroxy-2-methylpropyl)-2-oxo-2,3-dihydro-1,3-benzoxazol-5-yl]phenyl}ethyl]-1,4-oxazepane-2-carboxamide;
(2S)—N-[(1S)-1-Cyano-2-{4-[3-(2,2-difluoroethyl)-7-fluoro-2-oxo-2,3-dihydro-1,3-benzoxazol-5-yl]phenyl}ethyl]-1,4-oxazepane-2-carboxamide;
(2S)—N-[(1S)-1-Cyano-2-(4-{3-[2-(dimethylamino)ethyl]-2-oxo-2,3-dihydro-1,3-benzoxazol-5-yl}phenyl)ethyl]-1,4-oxazepane-2-carboxamide;
(2S)—N-{(1S)-1-Cyano-2-[4-(3,3-difluoro-1-methyl-2-oxo-2,3-dihydro-1H-indol-6-yl)phenyl]ethyl}-1,4-oxazepane-2-carboxamide;
(2S)—N-{(1S)-1-Cyano-2-[4-(7-fluoro-3-methyl-2-oxo-2,3-dihydro-1,3-benzoxazol-5-yl)phenyl]ethyl}-1,4-oxazepane-2-carboxamide;
(2S)—N-{(1S)-1-Cyano-2-[4-(3-ethyl-2-oxo-2,3-dihydro-1,3-benzoxazol-5-yl)phenyl]ethyl}-1,4-oxazepane-2-carboxamide;
(2S)—N-[(1S)-1-Cyano-2-{4-[3-(cyclopropylmethyl)-2-oxo-2,3-dihydro-1,3-benzoxazol-5-yl]phenyl}ethyl]-1,4-oxazepane-2-carboxamide;
(2S)—N-[(1S)-1-Cyano-2-{4-[3-(2-methoxyethyl)-2-oxo-2,3-dihydro-1,3-benzothiazol-5-yl]phenyl}ethyl]-1,4-oxazepane-2-carboxamide;
(2S)—N-[(1S)-1-Cyano-2-{4-[2-oxo-3-(propan-2-yl)-2,3-dihydro-1,3-benzoxazol-5-yl]phenyl}ethyl]-1,4-oxazepane-2-carboxamide;
(2S)—N-{(1S)-1-Cyano-2-[4-(4-methyl-3-oxo-3,4-dihydro-2H-1,4-benzoxazin-6-yl)phenyl]ethyl}-1,4-oxazepane-2-carboxamide;
(2S)—N-[(1S)-1-Cyano-2-{4-[3-(2-methoxyethyl)-2-oxo-2,3-dihydro-1,3-benzoxazol-5-yl]phenyl}ethyl]-1,4-oxazepane-2-carboxamide;
(2S)—N-{(1S)-1-Cyano-2-[4-(5-cyanothiophen-2-yl)phenyl]ethyl}-1,4-oxazepane-2-carboxamide;
(2S)—N-[(1S)-2-(4′-Carbamoyl-3′-fluorobiphenyl-4-yl)-1-cyanoethyl]-1,4-oxazepane-2-carboxamide;
(2S)—N-{(1S)-1-Cyano-2-[4-(1-methyl-2-oxo-1,2-dihydroquinolin-7-yl)phenyl]ethyl}-1,4-oxazepane-2-carboxamide;
(2S)—N-[(1S)-1-Cyano-2-{4-[2-oxo-3-(tetrahydro-2H-pyran-4-ylmethyl)-2,3-dihydro-1,3-benzoxazol-5-yl]phenyl}ethyl]-1,4-oxazepane-2-carboxamide;
(2S)—N-{(1S)-2-[4-(7-Chloro-3-methyl-2-oxo-2,3-dihydro-1,3-benzoxazol-5-yl)phenyl]-1-cyanoethyl}-1,4-oxazepane-2-carboxamide;
(2S)—N-[(1S)-1-Cyano-2-{4-[3-(2,2-difluoroethyl)-2-oxo-2,3-dihydro-1,3-benzoxazol-5-yl]phenyl}ethyl]-1,4-oxazepane-2-carboxamide;
(2S)—N-[(1S)-1-Cyano-2-{4-[2-oxo-3-(2,2,2-trifluoroethyl)-2,3-dihydro-1,3-benzoxazol-5-yl]phenyl}ethyl]-1,4-oxazepane-2-carboxamide;
(2S)—N-{(1S)-1-Cyano-2-[4-(3-methyl-2-oxo-2,3-dihydro-1,3-benzothiazol-5-yl)phenyl]ethyl}-1,4-oxazepane-2-carboxamide;
(2S)—N-{(1S)-1-Cyano-2-[4′-(methylsulfonyl)biphenyl-4-yl]ethyl}-1,4-oxazepane-2-carboxamide;
(2S)—N-{(1S)-2-[4′-(Azetidin-1-ylsulfonyl)biphenyl-4-yl]-1-cyanoethyl}-1,4-oxazepane-2-carboxamide;
(2S)—N-[(1S)-1-Cyano-2-(4′-fluorobiphenyl-4-yl)ethyl]-1,4-oxazepane-2-carboxamide;
(2S)—N-{(1S)-2-[4-(1,3-Benzothiazol-5-yl)phenyl]-1-cyanoethyl}-1,4-oxazepane-2-carboxamide;
(2S)—N-[(1S)-1-Cyano-2-(4′-cyanobiphenyl-4-yl)ethyl]-1,4-oxazepane-2-carboxamide;
and pharmaceutically acceptable salts thereof.

16. The method of claim 1 or 2, wherein the compound of Formula (I) is brensocatib; or a pharmaceutically acceptable salt thereof.

17. The method of claim 1 or 2, wherein the compound of Formula (I) is brensocatib.

18. The method of claim 1 or 3, wherein the compound of Formula (I) is (2S)—N-{(1R)-1-Cyano-2-[4-(3-methyl-2-oxo-2,3-dihydro-1,3-benzoxazol-5-yl)phenyl]ethyl}-1,4-oxazepane-2-carboxamide: 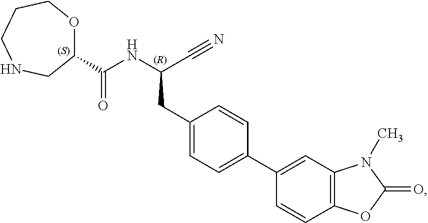
or a pharmaceutically acceptable salt thereof.

19. The method of claim 18, wherein the compound of Formula (I) is (2S)—N-{(1R)-1-Cyano-2-[4-(3-methyl-2-oxo-2,3-dihydro-1,3-benzoxazol-5-yl)phenyl]ethyl}-1,4-oxazepane-2-carboxamide:

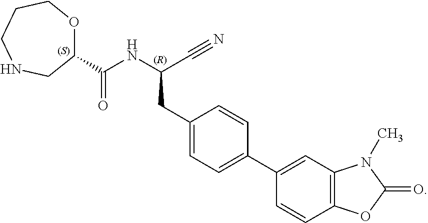

20. The method of claim 1 or 4, wherein the compound of Formula (I) is (2R)—N-{(1S)-1-Cyano-2-[4-(3-methyl-2-oxo-2,3-dihydro-1,3-benzoxazol-5-yl)phenyl]ethyl}-1,4-oxazepane-2-carboxamide: 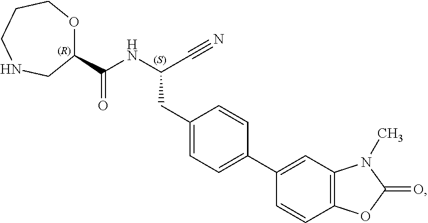
or a pharmaceutically acceptable salt thereof.

21. The method of claim 20, wherein the compound of Formula (I) is (2R)—N-{(1S)-1-Cyano-2-[4-(3-methyl-2-oxo-2,3-dihydro-1,3-benzoxazol-5-yl)phenyl]ethyl}-1,4-oxazepane-2-carboxamide:

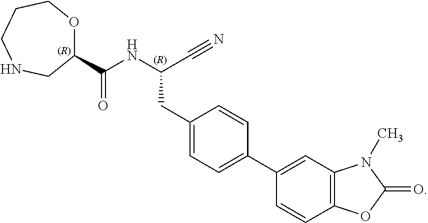

22. The method of claim 1 or 5, wherein the compound of Formula (I) is (2R)—N-{(1R)-1-Cyano-2-[4-(3-methyl-2-oxo-2,3-dihydro-1,3-benzoxazol-5-yl)phenyl]ethyl}-1,4-oxazepane-2-carboxamide: 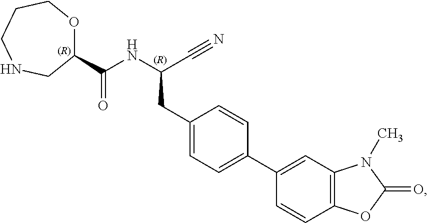
or a pharmaceutically acceptable salt thereof.

23. The method of claim 22, wherein the compound of Formula (I) is (2R)—N-{(1R)-1-Cyano-2-[4-(3-methyl-2-oxo-2,3-dihydro-1,3-benzoxazol-5-yl)phenyl]ethyl}-1,4-oxazepane-2-carboxamide:

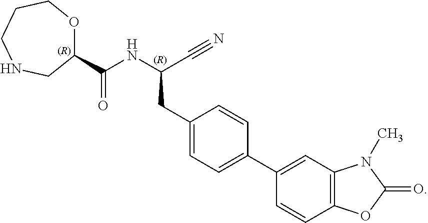

24. The method of claim 1, wherein the composition comprises a mixture of brensocatib, or a pharmaceutically acceptable salt thereof, and (2S)—N-{(1R)-1-Cyano-2-[4-(3-methyl-2-oxo-2,3-dihydro-1,3-benzoxazol-5-yl)phenyl]ethyl}-1,4-oxazepane-2-carboxamide: 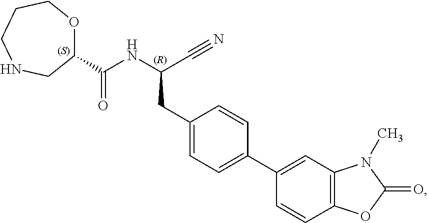
or a pharmaceutically acceptable salt thereof.

25. The method of claim 1, wherein the composition comprises a mixture of brensocatib, or a pharmaceutically acceptable salt thereof, and (2R)—N-{(1S)-1-Cyano-2-[4-(3-methyl-2-oxo-2,3-dihydro-1,3-benzoxazol-5-yl)phenyl]ethyl}-1,4-oxazepane-2-carboxamide: 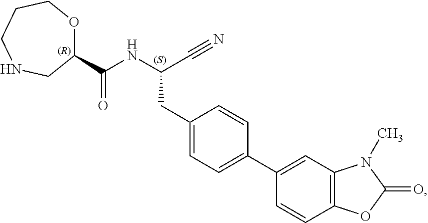
or a pharmaceutically acceptable salt thereof.

26. The method of claim 1, wherein the composition comprises a mixture of brensocatib, or a pharmaceutically acceptable salt thereof, and (2R)—N-{(1R)-1-Cyano-2-[4-(3-methyl-2-oxo-2,3-dihydro-1,3-benzoxazol-5-yl)phenyl]ethyl}-1,4-oxazepane-2-carboxamide: 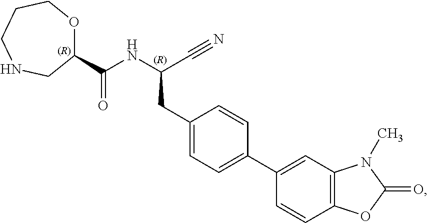
or a pharmaceutically acceptable salt thereof.

27. The method of any one of claims 1-26, wherein the composition comprises a pharmaceutically acceptable adjuvant, diluent or carrier.

28. The method of any one of claims 1-27, wherein the composition comprises:
(a) from about 1 to about 30 wt % of the compound of Formula (I), or a pharmaceutically acceptable salt thereof,
(b) from about 55 to about 75 wt % of a pharmaceutical diluent,
(c) from about 15 to about 25 wt % of a compression aid,
(d) from about 3 to about 5 wt % of a pharmaceutical disintegrant,
(e) from about 0.00 to about 1 wt % of a pharmaceutical glidant; and
(f) from about 2 to about 6 wt % of a pharmaceutical lubricant, wherein the component weights add up to 100 wt %.

29. The method of claim 28, wherein the pharmaceutical lubricant is glycerol behenate.

30. The method of claim 28 or 29, wherein the pharmaceutical diluent is microcrystalline cellulose.

31. The method of any one of claims 28-30, wherein the compression aid is dibasic calcium phosphate dihydrate.

32. The method of any one of claims 28-31, wherein the pharmaceutical disintegrant is sodium starch glycolate.

33. The method of any one of claims 28-32, wherein the pharmaceutical glidant is silicon dioxide.

34. The method of any one of claims 28-33, wherein the composition is in tablet form.

35. The method of claim 34, wherein the composition further comprises a tablet coating.

36. The method of any one of claims 28-35, wherein the compound of Formula (I) is present at about 3 to about 10 wt % of the total weight of the pharmaceutical composition.

37. The method of claim 36, wherein the pharmaceutical lubricant is glycerol behenate and the glycerol behenate is present at about 2.5 to about 4.5 wt % of the total weight of the composition.

38. The method of claim 36 or 37, wherein the pharmaceutical glidant is silicon dioxide and the silicon dioxide is present at about 0.05 to about 0.25 wt % of the total weight of the composition.

39. The method of any one of claims 36-38, wherein the pharmaceutical disintegrant is sodium starch glycolate and the sodium starch glycolate is present at about 3.5 to about 4.5 wt % of the total weight of the composition.

40. The method of any one of claims 36-39, wherein the compression aid is dibasic calcium phosphate dihydrate and the dibasic calcium phosphate dihydrate is present at about 18 to about 22 wt % of the total weight of the composition.

41. The method of any one of claims 36-40, wherein the pharmaceutical diluent is microcrystalline cellulose and the microcrystalline cellulose is present at about 55 to about 70 wt % of the total weight of the composition.

42. The method of any one of claims 1-41, wherein the compound of Formula (I) is present at from about 5 mg to about 70 mg in the composition.

43. The method of any one of claims 1-41, wherein the compound of Formula (I) is present at from about 10 mg to about 40 mg in the composition.

44. The method of any one of claims 1-41, wherein the compound of Formula (I) is present at about 10 mg in the composition.

45. The method of any one of claims 1-41, wherein the compound of Formula (I) is present at about 25 mg in the composition.

46. The method of any one of claims 1-41, wherein the compound of Formula (I) is present at about 40 mg in the composition.

47. The method of any one of claims 1-41, wherein the compound of Formula (I) is present at about 65 mg in the composition.

48. The method of any one of claims 1-47, wherein administering comprises oral administration.

49. The method of any one of claims 1-48, wherein administering to the patient is carried out one time daily during the administration period.

50. The method of any one of claims 1-48, wherein administering to the patient is carried out two times daily during the administration period.

51. The method of any one of claims 1-48, wherein administering to the patient is carried out every other day during the administration period.

52. The method of any one of claims 1-48, wherein administering to the patient is carried out every third day during the administration period.

53. The method of any one of claims 1-52, wherein the administration period is at least 1 month.

54. The method of any one of claims 1-53, wherein the administration period is from about 1 month to about 12 months.

55. The method of any one of claims 1-53, wherein the administration period is from about 6 months to about 24 months.

56. The method of any one of claims 1-53, wherein the administration period is from about 6 months to about 18 months.

57. The method of any one of claims 1-53, wherein the administration period is from about 6 months to about 15 months.

58. The method of any one of claims 1-53, wherein the administration period is about 6 months.

59. The method of any one of claims 1-53, wherein the administration period is about 12 months.

60. The method of any one of claims 1-53, wherein the administration period is about 18 months.

61. The method of any one of claims 1-53, wherein the administration period is about 24 months.

62. The method of any one of claims 1-53, wherein the administration period is from about 2 years to about 20 years.

63. The method of any one of claims 1-53, wherein the administration period is from about 5 years to about 15 years.

64. The method of any one of claims 1-53, wherein the administration period is from about 5 years to about 10 years.

65. The method of any one of claims 1-53, wherein the administration period is about 3 years.

66. The method of any one of claims 1-53, wherein the administration period is about 4 years.

67. The method of any one of claims 1-53, wherein the administration period is about 5 years.

68. The method of any one of claims 1-53, wherein the administration period is about 10 years.

69. The method of any one of claims 1-53, wherein the administration period is about 15 years.

70. The method of any one of claims 1-53, wherein the administration period is about 20 years.

71. The method of any one of claims 1-70, wherein treating comprises improving the lung function of the patient, as compared to the lung function of the patient prior to the administration period.

72. The method of claim 71, wherein the improving the lung function of the patient comprises increasing the patient's forced expiratory volume in one second (FEV₁) compared to the patient's FEV₁ prior to the administration period.

73. The method of claim 72, wherein the patient's FEV₁ is increased by about 5%, by about 10%, by about 15%, by about 20%, by about 25%, by about 30%, by about 35%, by about 40%, by about 45%, or by about 50%.

74. The method of claim 72, wherein the patient's FEV₁ is increased by at least about 5%, by at least about 10%, by at least about 15%, by at least about 20%, by at least about 25%, by at least about 30%, by at least about 35%, by at least about 40%, by at least about 45%, or by at least about 50%.

75. The method of claim 72, wherein the patient's FEV₁ is increased by about 5% to about 50%, by about 5% to about 40%, by about 5% to about 30%, by about 5% to about 20%, by about 10% to about 50%, by about 15% to about 50%, by about 20% to about 50%, or by about 25% to about 50%.

76. The method of claim 72, wherein the patient's FEV₁ is increased at least about 5%.

77. The method of claim 72, wherein the patient's FEV₁ is increased about 5% to about 50%, or about 10% to about 50%, or about 15% to about 50%.

78. The method of any one of claims 72-77, wherein the patient's FEV₁ is increased about 25 mL to about 500 mL.

79. The method of any one of claims 72-77, wherein the patient's FEV₁ is increased about 25 mL to about 250 mL.

80. The method of any one of claims 72-79, wherein the increasing the patient's FEV₁ is increasing the patient's pre-bronchodilator FEV₁.

81. The method of any one of claims 72-79, wherein the increasing the patient's FEV₁ is increasing the patient's post-bronchodilator FEV₁.

82. The method of any one of claims 71-81, wherein the improving the lung function of the patient comprises increasing the patient's percent predicted forced expiratory volume in one second (ppFEV₁) compared to the patient's ppFEV₁ prior to the administration period.

83. The method of claim 82, wherein the patient's ppFEV₁ is increased by about 1%, by about 2%, by about 3%, by about 4%, by about 5%, by about 6%, by about 7%, by about 8%, by about 9%, by about 10%, by about 11%, by about 12%, by about 13%, by about 14%, by about 15%, by about 16%, by about 17%, by about 18%, by about 19%, by about 20%, by about 25%, by about 30%, by about 35%, by about 40%, by about 45%, by about 50%, by about 55%, by about 60%, by about 65%, by about 70%, by about 75%, by about 80%, by about 85%, or by about 90%.

84. The method of claim 82, wherein the patient's ppFEV₁ is increased by at least about 5%, by at least about 10%, by at least about 15%, by at least about 20%, by at least about 25%, by at least about 30%, by at least about 35%, by at least about 40%, by at least about 45%, or by at least about 50%.

85. The method of claim 82, wherein the patient's ppFEV₁ is increased by about 5% to about 50%, by about 5% to about 40%, by about 5% to about 30%, by about 5% to about 20%, by about 10% to about 50%, by about 15% to about 50%, by about 20% to about 50%, or by about 25% to about 50%.

86. The method of claim 82, wherein the patient's ppFEV₁ is increased at least about 5%.

87. The method of claim 82, wherein the patient's ppFEV₁ is increased about 5% to about 50%, about 10% to about 50%, or about 15% to about 50%.

88. The method of any one of claims 82-87, wherein increasing the patient's ppFEV₁ is increasing the patient's pre-bronchodilator ppFEV₁.

89. The method of any one of claims 82-87, wherein increasing the patient's ppFEV₁ is increasing the patient's post-bronchodilator ppFEV₁.

90. The method of any one of claims 82-89, wherein the patient's ppFEV₁ is about 40% or more prior to the administration period.

91. The method of claim 90, wherein the patient's ppFEV₁ is about 50% or more prior to the administration period.

92. The method of claim 90, wherein the patient's ppFEV₁ is about 60% or more prior to the administration period.

93. The method of claim 90, wherein the patient's ppFEV₁ is about 70% or more prior to the administration period.

94. The method of claim 90, wherein the patient's ppFEV₁ is from about 40% to about 90% prior to the administration period.

95. The method of claim 90, wherein the patient's ppFEV₁ is from about 40% to about 80% prior to the administration period.

96. The method of claim 90, wherein the patient's ppFEV₁ is from about 50% to about 80% prior to the administration period.

97. The method of claim 90, wherein the patient's ppFEV₁ is from about 50% to about 70% prior to the administration period.

98. The method of any one of claims 71-97, wherein the improving the lung function of the patient comprises increasing the patient's forced vital capacity (FVC) compared to the patient's FVC prior to the administration period.

99. The method of claim 98, wherein the patient's FVC is increased by about 1%, by about 2%, by about 3%, by about 4%, by about 5%, by about 6%, by about 7%, by about 8%, by about 9%, by about 10%, by about 11%, by about 12%, by about 13%, by about 14%, by about 15%, by about 16%, by about 17%, by about 18%, by about 19%, by about 20%, by about 25%, by about 30%, by about 35%, by about 40%, by about 45%, by about 50%, by about 55%, by about 60%, by about 65%, by about 70%, by about 75%, by about 80%, by about 85%, or by about 90%.

100. The method of claim 98, wherein the patient's FVC is increased by at least about 5%, by at least about 10%, by at least about 15%, by at least about 20%, by at least about 25%, by at least about 30%, by at least about 35%, by at least about 40%, by at least about 45%, or by at least about 50%.

101. The method of claim 98, wherein the patient's FVC is increased by about 5% to about 50%, by about 5% to about 40%, by about 5% to about 30%, by about 5% to about 20%, by about 10% to about 50%, by about 15% to about 50%, by about 20% to about 50%, or by about 25% to about 50%.

102. The method of any one of claims 98-101, wherein the increasing the patient's FVC is increasing the patient's pre-bronchodilator FVC.

103. The method of any one of claims 98-101, wherein the increasing the patient's FVC is increasing the patient's post-bronchodilator FVC.

104. The method of any one of claims 71-103, wherein the improving the lung function of the patient comprises increasing the patient's forced expiratory flow between 25% and 75% of FVC (FEF_{(25-75%)}) compared to the patient's FEF_{(25-75%)} prior to the administration period.

105. The method of claim 104, wherein the patient's FEF_{(25-75%)} is increased by about 1%, by about 2%, by about 3%, by about 4%, by about 5%, by about 6%, by about 7%, by about 8%, by about 9%, by about 10%, by about 11%, by about 12%, by about 13%, by about 14%, by about 15%, by about 16%, by about 17%, by about 18%, by about 19%, by about 20%, by about 25%, by about 30%, by about 35%, by about 40%, by about 45%, by about 50%, by about 55%, by about 60%, by about 65%, by about 70%, by about 75%, by about 80%, by about 85%, or by about 90%.

106. The method of claim 104, wherein the patient's FEF_{(25-75%)} is increased by at least about 5%, by at least about 10%, by at least about 15%, by at least about 20%, by at least about 25%, by at least about 30%, by at least about 35%, by at least about 40%, by at least about 45%, or by at least about 50%.

107. The method of claim 104, wherein the patient's FEF_{(25-75%)} is increased by about 5% to about 50%, by about 5% to about 40%, by about 5% to about 30%, by about 5% to about 20%, by about 10% to about 50%, by about 15% to about 50%, by about 20% to about 50%, or by about 25% to about 50%.

108. The method of any one of claims 104-107, wherein the increasing the patient's FEF_{(25-75%)} is increasing the patient's pre-bronchodilator FEF_{(25-75%)}.

109. The method of any one of claims 104-107, wherein the increasing the patient's FEF_{(25-75%)} is increasing the patient's post-bronchodilator FEF_{(25-75%)}.

110. The method of any one of claims 71-109, wherein the improving the lung function of the patient comprises increasing the patient's peak expiratory flow rate (PEFR) compared to the patient's PEFR prior to the administration period.

111. The method of claim 110, wherein the patient's PEFR is increased by about 1%, by about 2%, by about 3%, by about 4%, by about 5%, by about 6%, by about 7%, by about 8%, by about 9%, by about 10%, by about 11%, by about 12%, by about 13%, by about 14%, by about 15%, by about 16%, by about 17%, by about 18%, by about 19%, by about 20%, by about 25%, by about 30%, by about 35%, by about 40%, by about 45%, by about 50%, by about 55%, by about 60%, by about 65%, by about 70%, by about 75%, by about 80%, by about 85%, or by about 90%.

112. The method of claim 110, wherein the patient's PEFR is increased by at least about 5%, by at least about 10%, by at least about 15%, by at least about 20%, by at least about 25%, by at least about 30%, by at least about 35%, by at least about 40%, by at least about 45%, or by at least about 50%.

113. The method of claim 110, wherein the patient's PEFR is increased by about 5% to about 50%, by about 5% to about 40%, by about 5% to about 30%, by about 5% to about 20%, by about 10% to about 50%, by about 15% to about 50%, by about 20% to about 50%, or by about 25% to about 50%.

114. The method of any one of claims 110-113, wherein the increasing the patient's PEFR is increasing the patient's pre-bronchodilator PEFR.

115. The method of any one of claims 110-113, wherein the increasing the patient's PEFR is increasing the patient's post-bronchodilator PEFR.

116. The method of any one of claims 1-115, wherein treating comprises improving the patient's quality of life (QOL) assessed by the cystic fibrosis questionnaire-revised (CFQ-R), as compared to the patient's QOL assessed by the CFQ-R prior to the administration period.

117. The method of claim 116, wherein the QOL is assessed by a respiratory domain score of the CFQ-R.

118. The method of any one of claims 1-117, wherein treating further comprises decreasing a sputum concentration of an active neutrophil serine protease (NSP) in the patient, as compared to the patient's active NSP sputum concentration prior to the administration period.

119. The method of claim 118, wherein the patient's active NSP sputum concentration is decreased by about 1%, about 5%, about 10%, about 20%, about 25%, about 30%, about 40%, about 50%, about 60%, about 70%, or about 80%.

120. The method of claim 118, wherein the patient's active NSP sputum concentration is decreased by at least about 10%, at least about 20%, at least about 30%, at least about 40%, at least about 50%, at least about 60%, at least about 70%, or at least about 80%.

121. The method of any one of claims 1-120, wherein treating further comprises decreasing a concentration of an active neutrophil serine protease (NSP) in the blood of the patient, as compared to the patient's active NSP blood concentration prior to the administration period.

122. The method of claim 121, wherein the patient's active NSP blood concentration is decreased by about 1%, about 5%, about 10%, about 20%, about 25%, about 30%, about 40%, about 50%, about 60%, about 70%, or about 80%.

123. The method of claim 121, wherein the patient's active NSP blood concentration is decreased by at least about 10%, at least about 20%, at least about 30%, at least about 40%, at least about 50%, at least about 60%, at least about 70%, or at least about 80%.

124. The method of any one of claims 118-123, wherein the active NSP is active neutrophil elastase (NE).

125. The method of any one of claims 118-123, wherein the active NSP is active proteinase 3 (PR3).

126. The method of any one of claims 118-123, wherein the active NSP is active cathepsin G (CatG).

127. The method of any one of claims 1-126, wherein the patient has previously been treated with a cystic fibrosis transmembrane conductance regulator (CFTR) modulator, and the treating further comprises administering the CFTR modulator to the patient.

128. The method of any one of claims 1-126, wherein the patient has not previously been treated with a cystic fibrosis transmembrane conductance regulator (CFTR) modulator, and the treating further comprises administering a CFTR modulator to the patient.

129. The method of claim 127 or 128, wherein the CFTR modulator is one selected from the group consisting of ivacaftor, lumacaftor, tezacaftor, elexacaftor, and a combination thereof.

130. The method of any one of claims 1-126, wherein the patient has not previously been treated with a CFTR modulator, and the method excludes administering a CFTR modulator to the patient.

131. The method of any one of claims 1-130, wherein treating further comprises administering an antibiotic to the patient.

132. The method of claim 131, wherein the antibiotic is selected from the group consisting of an aminoglycoside, aztreonam, a carbapenem, a cephalosporin, clofazimine, colistimethate, ethambutol, a lincosamide, a macrolide, an oxazolidinone, a penicillin, a quinolone, a rifamycin, a sulfa, a tetracycline, vancomycin, and a combination thereof.

133. The method of claim 131, wherein the antibiotic is selected from the group consisting of amikacin, aztreonam, colistimethate, gentamicin, tobramycin, or a combination thereof.

134. The method of claim 133, wherein the antibiotic is administered to the patient by inhalation.

135. The method of any one of claims 1-134, wherein treating further comprises decreasing a bacterial infection in the lung of the patient, as compared to the bacterial infection in the lung of the patient prior to the administration period.

136. The method of claim 135, wherein the bacterial infection comprises a Pseudomonas infection.

137. The method of claim 136, wherein the Pseudomonas infection comprises Pseudomonas aeruginosa infection.

138. The method of any one of claims 135-137, wherein the bacterial infection comprises Staphylococcus aureus infection.

139. The method of claim 138, wherein the Staphylococcus aureus infection is a methicillin-resistant Staphylococcus aureus (MRSA) infection.

140. The method of any one of claims 135-139, wherein the bacterial infection comprises Haemophilus influenzae infection.

141. The method of any one of claims 135-140, wherein the bacterial infection comprises Stenotrophomonas maltophilia infection.

142. The method of any one of claims 135-141, wherein the bacterial infection comprises Burkholderia cepacia complex infection.

143. The method of any one of claims 135-142, wherein the bacterial infection comprises Burkholderia cenocepacia infection.

144. The method of any one of claims 135-143, wherein the decreasing the bacterial infection in the lung of the patient comprises decreasing a number of colony forming units of the bacteria present in the patient's sputum, as compared to a number of colony forming units of the bacteria present in the patient's sputum prior to the administration period.

145. The method of claim 144, wherein the number of colony forming units of the bacteria present in the patient's sputum is decreased about 1%, about 5%, about 10%, about 20%, about 25%, about 30%, about 40%, about 50%, about 60%, about 70%, about 80%, or about 90%.

146. The method of claim 144, wherein the number of colony forming units of the bacteria present in the patient's sputum is decreased at least about 10%, at least about 20%, at least about 30%, at least about 40%, at least about 50%, at least about 60%, at least about 70%, at least about 80%, or at least about 90%.

147. The method of claim 144, wherein the number of colony forming units of the bacteria present in the patient's sputum is decreased by about 5% to about 50%, by about 5% to about 40%, by about 5% to about 30%, by about 5% to about 20%, by about 10% to about 50%, by about 15% to about 50%, by about 20% to about 50%, or by about 25% to about 50%.

148. (2R)—N-{(1R)-1-Cyano-2-[4-(3-methyl-2-oxo-2,3-dihydro-1,3-benzoxazol-5-yl)phenyl]ethyl}-1,4-oxazepane-2-carboxamide: 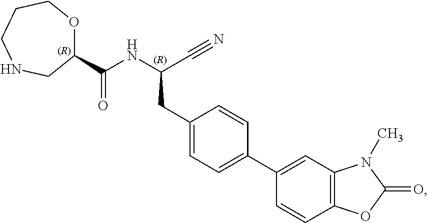
or a pharmaceutically acceptable salt thereof.

149. (2S)—N-{(1R)-1-Cyano-2-[4-(3-methyl-2-oxo-2,3-dihydro-1,3-benzoxazol-5-yl)phenyl]ethyl}-1,4-oxazepane-2-carboxamide: 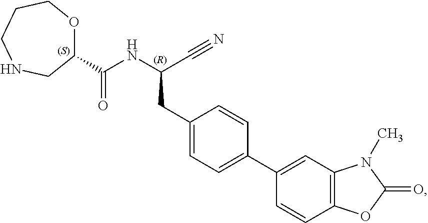
or a pharmaceutically acceptable salt thereof.

150. (2R)—N-{(1S)-1-Cyano-2-[4-(3-methyl-2-oxo-2,3-dihydro-1,3-benzoxazol-5-yl)phenyl]ethyl}-1,4-oxazepane-2-carboxamide: 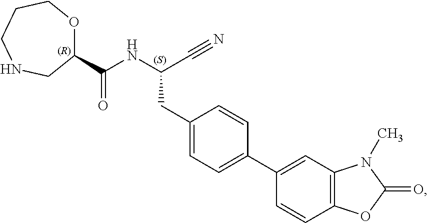
or a pharmaceutically acceptable salt thereof.

151. A mixture of brensocatib, or a pharmaceutically acceptable salt thereof, and (2R)—N-{(1R)-1-Cyano-2-[4-(3-methyl-2-oxo-2,3-dihydro-1,3-benzoxazol-5-yl)phenyl]ethyl}-1,4-oxazepane-2-carboxamide: 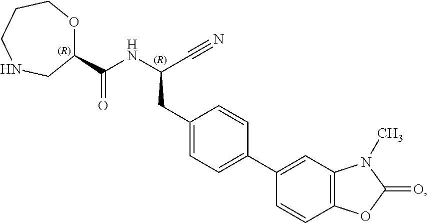
or a pharmaceutically acceptable salt thereof.

152. A mixture of brensocatib, or a pharmaceutically acceptable salt thereof, and (2S)—N-{(1R)-1-Cyano-2-[4-(3-methyl-2-oxo-2,3-dihydro-1,3-benzoxazol-5-yl)phenyl]ethyl}-1,4-oxazepane-2-carboxamide: 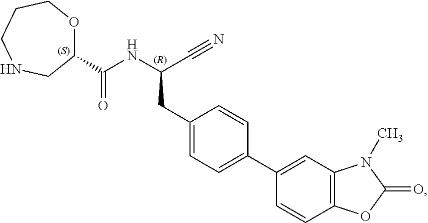
or a pharmaceutically acceptable salt thereof.

153. A mixture of brensocatib, or a pharmaceutically acceptable salt thereof, and (2R)—N-{(1S)-1-Cyano-2-[4-(3-methyl-2-oxo-2,3-dihydro-1,3-benzoxazol-5-yl)phenyl]ethyl}-1,4-oxazepane-2-carboxamide: 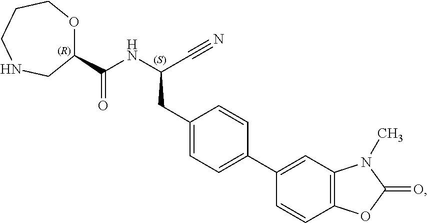
or a pharmaceutically acceptable salt thereof.
